(19) United States
(12) Patent Application Publication
(10) Pub. No.: US 2016/0296449 A1
(43) Pub. Date: Oct. 13, 2016

(54) Semi-Permanent Hair Straightening Composition And Method

(71) Applicant: LUBRIZOL ADVANCED MATERIALS, INC., Cleveland, OH (US)

(72) Inventors: Murat Kadir, Brecksville, OH (US); Neil Howard, Hudson, OH (US); Denise W. Rafferty, Sagamore Hills, OH (US); Joseph A. Zellia, Barberton, OH (US); Claudio Ribeiro, Sao Paulo (BR); Rebeca Dias, Sao Paulo (BR); Luciene Baptista Bastos, Sao Paulo (BR)

(21) Appl. No.: 15/035,049

(22) PCT Filed: Nov. 6, 2014
(86) PCT No.: PCT/US14/64217

§ 371 Date: May 6, 2016

Related U.S. Application Data
(60) Provisional application No. 61/901,528, filed on Nov. 8, 2013.

(51) Int. Cl. A61K 8/49; A45D 7/06; A61Q 5/12; A45D 2/00; A61Q 5/06; A61K 8/43

(52) U.S. Cl. CPC A61K 8/4973; A61Q 5/06; A61K 8/43; A61Q 5/12; A61K 8/4913; A61K 8/4953; A45D 2/001; A45D 7/06; A61K 2800/30; A61K 2800/882

(57) ABSTRACT

A composition and a process for straightening hair are disclosed. The process includes coating keratin fibers with a composition comprising a thermally-activated agent and a guanidine moiety containing adjuvant compound and contacting the coated keratin fibers with a heating device at a temperature of at least 185 C for sufficient time to modify the keratin fibers.

### BACKGROUND

The exemplary embodiment relates to a method for straightening hair by application of heat and to a composition which can be activated by heat. The composition comprises: a) at least one heterocyclic compound containing two heteroatoms selected from nitrogen and oxygen in a 5 to 8-membered ring, such as a cyclic alkylene carbonate and b) at least one hair straightening adjuvant compound containing a guanidine moiety.

Various methods are available for semi-permanent and permanent straightening of hair which can maintain the hair in a modified state for several washes. Many of the permanent treatment methods make use of harsh chemicals which can be hazardous to those performing and receiving the treatment or which give off unpleasant odors. Some of these methods are based on cleavage of the cystine disulfide covalent bonds that are present in keratin. In one method, the disulfide bonds are first broken with a reducing agent. Then a fixative, such as hydrogen peroxide, is applied while the hair is under tension to reconstitute the disulfide bonds in a different arrangement. The peroxide can cause damage to the hair and scalp. Other methods use a hydroxide base which replaces the disulfide bonds (—CH₂—S—S—CH₂—) with lanthionine bonds (—CH₂—S—CH₂—) in a two stage process using a hydroxide ion in the first step and a thiol group in the second. The hydroxide is used at fairly high concentrations and can cause scalp burning and irritation or damage of the eyes and nose. Thiols leave the hair with an unpleasant odor and can lead to degradation of the hair fibers.

Semi-permanent methods use a cross-linking agent to form bonds while the hair is heated. Formaldehyde, for example, is used in many commercial straightening products, either as an ingredient of the composition or as a reaction product that is formed on heating of the hair. However, it can cause allergic reactions to the skin, eyes and lungs, can cause severe damage to the eyes, and poses other health risks. The use of formaldehyde in hair straightening compositions has been discouraged or banned in some countries but it remains in widespread use, due to its effectiveness. Relaxing compositions including a denaturing agent such as urea have also been proposed. However, while urea is less caustic than a hydroxide-based composition, it can decompose to ammonia and poisonous isocyanic acid at the high temperatures used for thermal hair straightening.

### BRIEF DESCRIPTION

In accordance with one aspect of an exemplary embodiment of the disclosed technology, the technology relates to a thermally-activated composition for straightening hair comprising: (a) at least one hair straightening agent; (b) at least one hair straightening adjuvant; and optionally, (c) at least one cosmetically acceptable excipient.

In accordance with one aspect of an exemplary embodiment of the disclosed technology, the technology relates to a process for straightening keratin fibers comprising: i) coating keratin fibers with a composition comprising: a thermally-activated composition for straightening hair including: (a) at least one heterocyclic compound containing two heteroatoms, at least one of said heteroatoms being oxygen and the other being selected from nitrogen and oxygen in a 5 to 8-membered ring; (b) at least one hair straightening adjuvant compound containing a guanidine moiety containing compound; and optionally (c) at least one cosmetically acceptable excipient, and ii) contacting the coated keratin fibers with a heating device at a temperature of at least 185° C. for sufficient time to modify the keratin fibers.

It has been discovered that the combination of the at least one aforementioned heterocyclic compound containing two heteroatoms at least one of said heteroatoms being oxygen and the other being selected from nitrogen and oxygen in a 5 to 8-membered ring with the at least one guanidine moiety containing adjuvant compound provides a hair straightening formulation with synergistic efficacy in terms of longer hold times, lower hair straightening agent loading amounts, lower treating temperatures, shortened contact times between the initial application of the hair straightening composition to the hair and the heat treatment step, and the ability to maintain hold over multiple shampoo wash cycles, when compared to hair straightening formulations containing the said heterocyclic compound alone.

The at least one hair straightening agent (a) is a compound represented by general Formula (I) as follows:

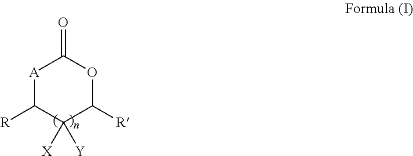

where:
A is selected from NH and O;
R and R′ are independently selected from H, CH₃, CH₂CH₃ and CH₂OH;
X and Y are independently selected from H and alkyl; and
n=0, or 1, or 2, or 3.

In one embodiment, the at least one hair straightening adjuvant (b) is a guanidine derivative and/or the organic and inorganic salts thereof selected from a compound containing a guanidine moiety represented by the radical:

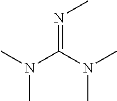

The open bond lines in the above moiety represent a linkage to another atom, functional group and/or radical such that the valance of each nitrogen atom in the guanidine moiety is satisfied. The guanidine moiety can be present in a liner or a cyclic guanidine moiety containing compound (guanidine derivative). In one embodiment the guanidine moiety containing compound can be represented by Formula (II) as follows:

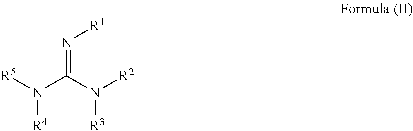

where:
R¹, R², R³, R⁴ and R⁵ independently represent a hydrogen atom; linear or branched C₁-C₄ alkyl; linear or branched C₂ to C₄ alkenyl, wherein said linear or branched C₁-C₄ alkyl and said linear or branched C₂ to C₄ alkenyl can optionally be substituted with one or two substituents selected from hydroxyl, amino, dimethylamino, carboxyl, carboxamide, N-methylcarboxamide and SO₃H; and any two of R¹, R², R³, R⁴ and R⁵ together with the nitrogen atom(s) to which they are attached form a 4 to 9 membered monocyclic or bicyclic ring containing 2 to 4 carbon atoms, optionally containing one or two (ring) heteroatom(s) selected from C(O), N, O and S, and said carbon atom(s) being optionally substituted with one or two substituent(s) selected from hydroxyl and amino; and when R¹, R², R³ and R⁴ represent a hydrogen atom, R⁵ can represent a radical selected from acetyl; chloroacetyl; carboxamide; N-methylcarboxamide; methoxy; ethoxy; 1,2,4-triazolyl; cyclopentyl; cyclohexyl; methoxycarbonyl; ethoxycarbonyl; C(O)CH═CHC(O)OH; phenyl; phenyl substituted with chlorine or hydroxyl; benzyl; thiazolidonyl; benzimidazolyl; benzoxazolyl; benzothiazolyl; or
C(═NH)—NR⁶R⁷ where R⁶ and R⁷ independently represent a hydrogen atom or a linear or branched C₁-C₄ alkyl radical, optionally substituted with one or 2 substituents selected from hydroxyl, amino, dimethylamino, carboxyl and carboxamide.

The thermally-activated hair straightening agent (a) and adjuvant (b) defined above can optionally comprise one or more cosmetically acceptable excipient(s) (c).

### DETAILED DESCRIPTION

Exemplary embodiments in accordance with the disclosed technology will be described. Various modifications, adaptations or variations of the exemplary embodiments described herein may become apparent to those skilled in the art as such are disclosed. It will be understood that all such modifications, adaptations or variations that rely upon the teachings of the disclosed technology, and through which these teachings have advanced the art, are considered to be within the scope and spirit of the presently disclosed technology.

The compositions, systems, and methods of the disclosed technology may suitably comprise, consist of, or consist essentially of the components, elements, steps, and process delineations described herein. The technology illustratively disclosed herein suitably may be practiced in the absence of any element which is not specifically disclosed herein.

Unless otherwise stated, all percentages, parts, and ratios expressed herein are based upon weight of the total compositions of the disclosed technology.

While overlapping weight ranges for the various ingredients that make up the disclosed thermally-activated hair straightening composition will be expressed for various embodiments of the disclosed technology, it should be readily apparent that the specific amount of each component in the composition will be selected from its disclosed range such that the desired amount of each component is adjusted so that the sum of all components in the composition totals 100 wt. %.

By “thermally-activated” or “thermally-activatable” it is meant that the hair straightening composition can be activated or is activatable by heat, as in a hair-straightening process, although the term is not intended to be considered as limiting the mode of operation of the hair straightening agent(s) and adjuvant(s) in the composition.

A “cosmetically acceptable excipient” is a non-toxic, non-irritating substance used as a diluent, carrier vehicle, and/or additive for components (a) and (b), which when mixed with the thermally-activated hair straightening agent(s) and adjuvant(s) of the disclosed technology provides a composition that is more suitably applied to the hair and/or imparts other desirable effects to the hair and/or scalp.

The terms “straight”, or “straightening”, or “straighten”, or “relaxing”, or relaxing and grammatical variations thereof mean to remove the curl from the hair or reduce the degree of curl of the hair. It also means changing the configuration of the hair or degree of curl of the hair to make the hair straighter.

The headings provided herein serve to illustrate, but not to limit the disclosed technology in any way or manner.

An exemplary embodiment of the disclosed technology relates to a thermally-activated semi-permanent hair straightening or hair relaxing composition and a method for its use. The exemplary composition includes: (a) at least one heterocyclic compound containing two heteroatoms at least one of said heteroatoms being oxygen and the other being selected from nitrogen and oxygen in a 5 to 8-membered ring; (b) at least one hair straightening adjuvant compound containing a guanidine moiety; and optionally (c) a cosmetically acceptable excipient.

In one exemplary embodiment, the hair straightening agent (a) is present in an amount ranging from about 10 to about 70 wt. % in a first aspect, from about 12 to about 50 wt. % in a second aspect, from about 15 to about 40 wt. % in a third aspect, from about 20 to about 35 wt. % in a fourth aspect, and from about 22 to about 30 wt. % in a fifth aspect (based on the total weight of the composition).

In one exemplary embodiment, the ratio of the hair straightening agent(s) to the hair straightening adjuvant(s) ranges from about 1.5 to 1 to about 10:1 by weight in a first aspect, from about 3:1 to about 9:1 in a second aspect, from about 5:1 to about 8:1 in a third aspect, and from about 6:1 to about 7:1 in a fourth aspect. In one exemplary embodiment, the ratio of the hair straightening agent(s) to the hair straightening adjuvant(s) is about 2:1, or about 3:1, or about 4:1, or about 5:1, or about 6:1, or about 7:1, or about 8:1, or about 9:1, or about 10:1.

In one exemplary embodiment, the disclosed technology relates to a system for straightening or relaxing hair. The system comprises at least two separate components including: i) a first hair straightening component (a) comprising at least one heterocyclic compound containing two heteroatoms at least one of said heteroatoms being oxygen and the other being selected from nitrogen and oxygen in a 5 to 8-membered ring; ii) a second component (b) comprising at least one hair straightening adjuvant selected from a guanidine moiety containing compound; and iii) said first component (a) and said second component (b) independent of one another optionally contain (c) a cosmetically acceptable excipient; wherein said first component (a) is devoid of said hair straightening adjuvant contained in (b) and said second component (b) is devoid of said hair straightening agent contained in (a); and wherein said component (a) and said component (b) are suitable for being mixed together to form a thermally-activatable hair straightening composition.

In one exemplary embodiment of the disclosed system (after mixing components (a) and (b) together), said hair straightening agent(s) is present in an amount ranging from about 10 to about 70 wt. % in a first aspect, from about 12 to about 50 wt. % in a second aspect, from about 15 to about 40 wt. % in a third aspect, from about 20 to about 35 wt. % in a fourth aspect, and from about 22 to about 30 wt. % (based on the total weight of the composition).

In one exemplary embodiment of the disclosed system (after mixing components (a) and (b) together), the ratio of said hair straightening agent(s) in (a) to said adjuvant(s) in (b) ranges from about 1.5 to 1 to about 10:1 by weight in a first aspect, from about 3:1 to about 9:1 in a second aspect, from about 5:1 to about 8:1 in a third aspect, and from about 6:1 to about 7:1 in a fourth aspect. In one exemplary embodiment, the ratio of the hair straightening agent(s) to the hair straightening adjuvant(s) is about 2:1, or about 3:1, or about 4:1, or about 5:1, or about 6:1, or about 7:1, or about 8:1, or about 9:1, or about 10:1.

In one exemplary embodiment, the disclosed technology relates to a kit for preparing a thermally-activatable composition for straightening or relaxing hair. The kit comprises at least two separate components including: i) a hair straightening component (a) contained in a first container, said component comprising at least one heterocyclic compound containing two heteroatoms at least one of said heteroatoms being oxygen and the other being selected from nitrogen and oxygen in a 5 to 8-membered ring; H) a hair straightening adjuvant component (b) contained in a second container, said component comprising at least one hair straightening adjuvant selected from a guanidine moiety containing compound; and iii) said first component (a) and said second component (b) independent of one another optionally contain (c) a cosmetically acceptable excipient; wherein said first component (a) is devoid of said hair straightening adjuvant contained in (b) and said second component (b) is devoid of said hair straightening agent contained in (a); and wherein said component (a) and said component (b) are suitable for being mixed together to form a thermally-activatable hair straightening composition.

In one exemplary embodiment of the disclosed kit (after mixing components (a) and (b) together), said hair straightening agent(s) is present in an amount ranging from about 10 to about 70 wt. % in a first aspect, from about 12 to about 50 wt. % in a second aspect, from about 15 to about 40 wt. % in a third aspect, from about 20 to about 35 wt. % in a fourth aspect, and from about 22 to about 30 wt. % in a fifth aspect (based on the total weight of the composition).

In one exemplary embodiment of the disclosed kit (after mixing components (a) and (b) together), the ratio of said hair straightening agent(s) in (a) to said adjuvant(s) in (b) ranges from about 1.5 to 1 to about 10:1 by weight in a first aspect, from about 3:1 to about 9:1 in a second aspect, from about 5:1 to about 8:1 in a third aspect, and from about 6:1 to about 7:1 in a fourth aspect. In one exemplary embodiment, the ratio of the hair straightening agent(s) to the hair straightening adjuvant(s) is about 2:1, or about 3:1, or about 4:1, or about 5:1, or about 6:1, or about 7:1, or about 8:1, or about 9:1, or about 10:1.

The individual components (a) and (b) or the mixed composition comprising components (a) and (b) can be in the form of a liquid, cream, mousse, gel, spray, lotion, pomade, and the like.

In one embodiment, the composition comprising components (a) and (b) is formulated into a cream base. The cream base is formulated with ingredients in such quantities so as to provide the cream with a level of viscosity that allows the cream to be easily applied to the hair in controllable volumes, without clumping, running or dripping. The cream should thick enough to keep the hair in a straightened position during the processing time, but not so thick as to make the cream unduly difficult to spread over the hair, and it should not be so thin as to drip from the hair onto the scalp. These creams can be made in the form of an emulsion, for example, based on glyceryl stearate, glycol stearate, self-emulsifying waxes, fatty alcohols, mineral oil, and petrolatum. Methods and ingredients to formulate cream bases are well-known in the art and are easily prepared by the skilled formulator.

In another embodiment, the disclosed technology relates to a method for straightening or relaxing keratin fibers, such as human hair. The method includes contacting keratin fibers with the exemplary composition in accordance with the disclosed technology to coat the fibers, maintaining the keratin fibers in contact with the composition for a sufficient contact time to effect straightening when the fibers are heated, and optionally drying the coated fibers to remove any moisture that may be present. In one embodiment, subsequent applying the composition to the keratin fibers and prior to applying heat to the coated fibers, the composition is allowed to remain in place for a sufficient contact time to permit the active hair straightening agent(s) to penetrate into the cortex of the fiber. The contact time can range from about 5 to about 60 minutes in one aspect, from about 10 to 50 minutes in another aspect, from about 15 to about 45 minutes in still another aspect, and from about 20 to about 30 minutes in a further aspect. Subsequent to the end of the contact time period and prior to applying heat, the coated fibers optionally are combed from root to tip to remove any excess coating (hair straightening composition) from the fibers. Subsequent to the optional combing step and prior to the heating step, the keratin fibers can be subjected to an optional smoothing step by blow drying the fibers while brushing the fibers from the root to the tip. The smoothed keratin fibers are contacted with a surface of a heating device, such as a flat iron, having a temperature of from 185 to 250° C. in one aspect, from about 190 to about 240° C. in another aspect, from about 200 to about 230° C. in a further aspect, and from about 205 to about 215° C. in a still further aspect for sufficient time to straighten or relax the keratin fibers, e.g., by reaching the glass transition temperature, which can vary, to some degree, depending on the moisture content in the fiber.

In one embodiment, the heating device is pulled through the hair fibers from the root to the tip for at least several repetitions. The heating device is pulled through the hair to be treated for at least 5 repetitions in one aspect, for at least 10 repetitions in another aspect, for at least 12 repetitions in still another aspect, for at least 15 repetitions in a further aspect, for at least 25 repetitions in a still further aspect.

In one embodiment, the heating device is pulled through the hair fibers at glide rate of about 1 cm/sec. in one aspect, of about 2 cm/sec. in another aspect, of about 2.5 cm/sec. in still another aspect, of about 3 cm/sec. in a further aspect, of about 3.5 cm/sec. in a still further aspect, and about 4 cm/sec. in an additional aspect. The glide rate is defined as the rate or speed at which the heating device is pulled through the length of the hair from the root end to the tip.

In embodiments where the pH of the hair straightening composition is in the alkaline region, subsequent to the thermal straightening or relaxing step, a neutralizing and conditioning masque optionally can be applied to the keratin fibers followed by thoroughly rinsing the fibers with warm water. Following the rinse, the fibers are towel dried and then smoothed with a blow drier and brush to achieve the desired final straightened or relaxed configuration. The straightening or relaxation generally results in an increase in the average length of the keratin fibers, as measured when a lock of the curly fibers is suspended from a support, of at least 5% or at least 10%, which can be maintained over several hair washing treatments (involving shampooing and drying the hair), such as at least 12, or at least 18, or at least 20, or at least 24, or at least 30, or at least 36, or at least 50 washes. The exemplary process is semi-permanent in that over time, the straightened fibers begin to return to their original, curly state. While the method can be used to form very straight hair, it can also be used to relax the hair from the original curly state in a process in which a semi-permanent wave is created.

In one embodiment, before the hair is treated with the compositions of the disclosed technology, the hair can be subjected to an optional shampooing step with a cleansing shampoo. Any suitable commercially available cleansing shampoo will suffice.

In one embodiment, before the hair is treated with the compositions of the disclosed technology, the hair can be shampooed with an alkaline cleansing shampoo that is formulated in the pH range of about 7.0 to about 9.5. While not wishing to be bound by theory it is thought that the alkaline pH opens up the cuticle of the hair shaft permitting easier penetration of the subsequently applied hair straightening active ingredients into the cortex of the keratin fiber.

### Thermally-Activated Hair Straightening Agent

The thermally-activated hair straightening agent used in the semi-permanent hair straightening composition and method of the disclosed technology is selected from at least one heterocyclic compound containing two heteroatoms at least one of said heteroatoms being oxygen and the other being selected from nitrogen and oxygen in a 5 to 8-membered ring represented by Formula (I):

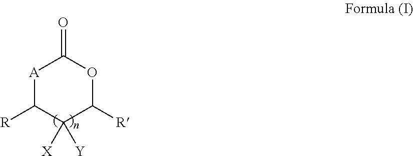

where:
A is selected from NH (nitrogen) and O (oxygen);
R and R′ are independently selected from H, CH₃, CH₂CH₃ and CH₂OH;
X and Y are independently selected from H and alkyl, such as a C₁-C₆ or C₁-C₃ alkyl group, such as CH₃; and
n=0 or 1, or 2, or 3.

In one embodiment, A=0. The structure thus represents an alkylene carbonate.

In one embodiment, at least one of R and R′ is H. In another embodiment, both R and R′ are H.

In one embodiment, n=0, i.e., the structure represents a five-membered ring of the general Formula (IA):

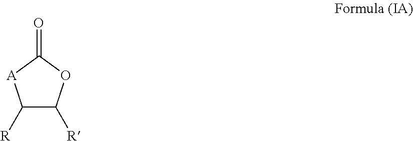

where A, R and R′ are as defined above.

As one example, the alkylene carbonate can be of the general Formula (IB):

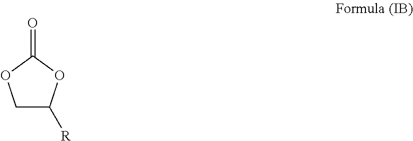

where R is as defined above.

Five-membered alkylene carbonates (1,3-dioxolan-2-ones), such as ethylene carbonate (“EC”, where R and R′ are H), propylene carbonate (“PC”, where R is CH₃ and R′ is H), butylene carbonate (“BC”, where R is CH₂CH₃ and R′ is H or where R is CH₃ and R′ is CH₃), and glycerol carbonate (“GC”, where R is CH₂OH and R′ is H) are exemplary thermally-activated agents useful herein.

In one embodiment, when n=1, at least one or both X and Y represent H.

Six-membered alkylene carbonates (1,3-dioxan-2-ones) useful herein include trimethylene carbonate (X and Y=H, R and R′=H). Exemplary thermally-activated agents where A is N include 2-oxazolidinone (R and R′ is H, n is 0) and derivatives thereof.

Exemplary thermally-activated agent(s) according to Formula (I) are small molecules (MW≦800 g/mol, or ≦600 g/mol, such as ≦200 g/mol) rather than oligomeric compounds or polymers (≧1000 g/mol). They are water compatible and stable, able to penetrate and react with keratinous materials, and can be activated by a flat or round iron at 210° C.

In one embodiment, compounds represented by Formula (I), such as the alkylene carbonates, are suited to an exemplary application as they generally have low toxicity and do not form toxic byproducts during the heating stage. They are also highly soluble in water at room temperature (25° C.), with ethylene carbonate being highly soluble (concentrations over 50 wt. % in water being readily feasible) and propylene carbonate being soluble in lesser amounts (up to about 20 wt. %).

In one embodiment, the thermally-activated agent(s) according to Formula (I) is/are present in the straightening composition at a concentration of at least 10 wt. %, or at least 12 wt. %, or at least 15 wt. %, or at least 18 wt. %, or at least 20 wt. %, or at least 22 wt. %, or at least 25 wt. %, or at least 27 wt. %, or at least 30 wt. %, and in some embodiments the thermally-activated agent is present in the straightening composition at a concentration ranging from about 12 wt. % to about 70 wt. % in one aspect, from about 12 wt. % to about 50 wt. % in another aspect, from about 15 wt. % to about 40 wt. % in still another aspect, from 20 wt. % to about 35 wt. % in a further aspect, and from about 22 to about 30 wt. % in a still further aspect (all based on the total weight of the straightening composition).

In one embodiment, the thermally-activated hair straightening agent is selected from ethylene carbonate (1,3-dioxolan-2-one), propylene carbonate, glycerol carbonate, trimethylene carbonate, 2-oxazolidinone and combinations thereof. In one embodiment, such thermally-activated agent(s) may suitably be present in the composition at a total concentration of 10-40 wt. %, or at least 12 wt. %, or at least 15 wt. %, or at least 18 wt. %, or at least 20 wt. %, or at least 22 wt. %, or at least 25 wt. %, or at least 27 wt. % or at least 30 wt. %, or at least 35 wt. % (all based on the total weight of the hair straightening composition).

In one embodiment, the thermally-activated agent includes a mixture of ethylene carbonate and propylene carbonate at a weight ratio of from about 95:5 to about 5:95 in one aspect, from about 90:10 to about 10:90 in a second aspect, from about 80:20 to about 20:80 in a third aspect, from about 75:25 to about 25:75 in a fourth aspect, and about 50:50 in a fifth aspect. At ambient temperatures (e.g., 20 to 25° C.) ethylene carbonate exists in the solid state and propylene carbonate exists in the liquid state. In such embodiments, the propylene carbonate may replace some or all of other solvents/diluents used in the composition given that ethylene carbonate has excellent solubility in propylene carbonate.

Alkylene carbonates suitable for use herein are available from Huntsman Corporation under the tradename JEFFSOL® alkylene carbonates. JEFFSOL ethylene carbonate is solid at room temperature. JEFFSOL EC-75, EC-50, and EC-25 are blends of JEFFSOL ethylene carbonate and propylene carbonate in the ratios of 75/25, 50/50 and 25/75 by weight, respectively. These blends are liquid at room temperature. As sold, these products are said to be at least 99.8% pure. Higher purity versions are also available.

U.S. Pat. No. 2,873,282 describes methods for making alkylene carbonates by reacting an alkylene oxide or compound thereof with carbon dioxide. U.S. Pat. No. 2,773,070 describes similar methods. Typically, an alkylammonium halide catalyst such as tetraethylammonium bromide is employed. Glycerol (glycerin) carbonate (GC) is available commercially and can be synthesized by the reaction of glycerin with a carbonate source such as phosgene, a dialkyl carbonate, or an alkylene carbonate; by reaction of glycerin with urea, carbon dioxide, and oxygen; or by reaction of carbon dioxide with glycidol.

### Thermally-Activated Hair Straightening Adjuvant

The thermally-activated hair straightening adjuvant used in the semi-permanent hair straightening composition and method of the disclosed technology is selected from at least one hair straightening adjuvant compound containing a guanidine moiety. In one embodiment, the at least one hair straightening adjuvant containing the guanidine moiety is a compound represented by general Formula (II) as follows:

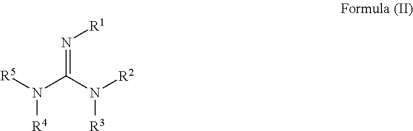

In one aspect, R¹, R², R³, R⁴ and R⁵ independently represent a hydrogen atom; linear or branched C₁ to C₈ alkyl; linear or branched C₂ to C₄ alkenyl; phenyl; benzyl; wherein said linear or branched C₁-C₄ alkyl (e.g., methyl, ethyl, propyl, butyl), said linear or branched C₂ to C₄ alkenyl, said phenyl and said benzyl groups can optionally be substituted with one or two substituents selected from halo (e.g, bromo, chloro, fluoro, iodo, and combinations thereof), amino, dimethylamino, carboxyl, hydroxyl, methyl, methoxy, methoxycarbonyl, carboxamide, N-methylcarboxamide and —SO₃H; and any two of R¹, R², R³, R⁴ and R⁵ together with the nitrogen atom(s) to which they are attached form a 4 to 9 membered monocyclic or bicyclic ring containing 2 to 4 carbon atoms, optionally containing one or two (ring) heteroatom(s) selected from C(O), N, O and S, and said carbon atom(s) being optionally substituted with one or two substituent(s) selected from hydroxyl and amino.

In one aspect in said Formula (II), R¹ represents a hydrogen atom, —C(CH₃)₃, —C(O)OC(CH₃)₃, or the radical:

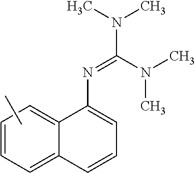

where the open bond line in the naphthalene moiety represents a covalent linkage to the nitrogen atom in Formula (II); and R², R³, R⁴, and R⁵ independently represent a hydrogen atom, a radical selected from amino, nitro; cyano; acetyl; chloroacetyl; carboxyl, carboxamide; N-methylcarboxamide; methoxy; ethoxy; 1,2,4-triazolyl; cyclopentyl; cyclohexyl; —C(O)CH═CHC(O)OH; —CH₂(CO)OCH₃; —CH₂C(O)OCH₂CH₃; —C(O)OC(CH₃)₃; thiazolidonyl; benzimidazolyl; benzoxazolyl; benzothiazolyl; —C(═NH)NH₂; —C(═NH)—NR⁶R⁷ where R⁶ and R⁷ independently represent a hydrogen atom or a linear or branched C₁-C₄ alkyl radical, optionally substituted with one or 2 substituents selected from hydroxyl, amino, dimethylamino, carboxyl and carboxamide;

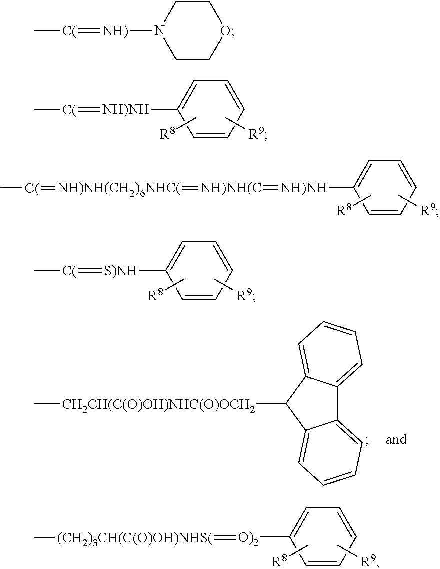

where R⁸ and R⁹ independently represent halo (e.g, bromo, chloro, fluoro, iodo, and combinations thereof), methyl, methoxy, trifluoromethyl, and trifluoromethoxy.

In one aspect in said Formula (II), R¹, R² and R³ represent a hydrogen atom; and R⁴ and R⁵ taken together with the nitrogen atom to which they are attached, represents a pyrrolidine, piperidine, pyrazole, morpholine, thiazolidone, benzimidazole, benzoxazole, benzothiazole, or 1,2,4-triazole ring, wherein said rings can be optionally substituted with 1 or 2 substituents selected from hydroxyl, amino and carboxyl groups.

In one aspect in said Formula (II), R¹ and R² represent a hydrogen atom, and R⁵ represents a hydrogen atom or methyl, R³ and R⁴ taken together can represent a divalent radical selected from —CH₂—CH₂— and —CH₂—O— and when taken together with the nitrogen atoms to which they are attached represent a 5 membered ring.

In one aspect in said Formula (II), R¹, R² and R³, R⁴ can be taken together with the nitrogen atoms to which they are attached to form a bicyclic fused ring structure represented by Formula (IIA):

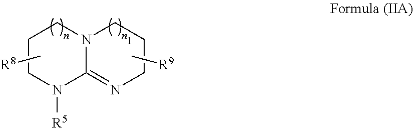

where R⁵ is as defined above; R¹⁰ and R¹¹ are optionally present, and when present one or two of R¹⁰ and R¹¹ can be bonded to the same or different ring carbon atoms and independently represent C₁-C₅ alkyl (e.g., methyl, ethyl, propyl, isopropyl, butyl, isobutyl, pentyl, neopentyl), hydroxyl, amino, dimethylamino, carboxyl, carboxamide, N-methylcarboxamide and SO₃H; and n and n₁ independently represent 0, 1 or 2.

In one aspect, the bicyclic fused ring can be represented by Formulas (IIB) to (IIF):

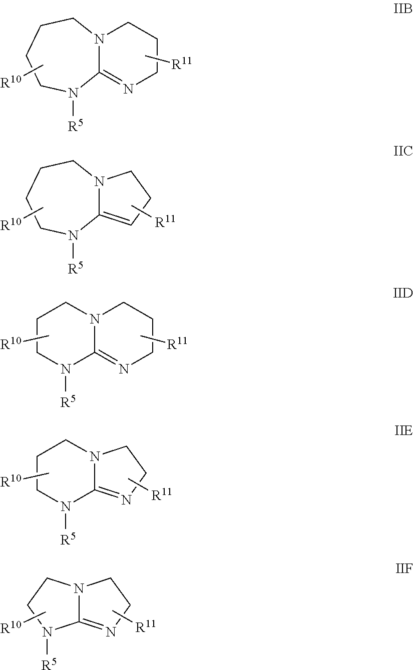

where R⁵, R¹⁰ and R¹¹ are as previously defined.

In one aspect, salts of the compounds represented by Formula (II) to Formula (IIF) can be utilized. Suitable salts include, but are not limited to the hydrochloride, nitrate, phosphate, sulfate, hemisulfate, sulfamate, acetate, carbonate, bicarbonate, and monohydrate salts of the guanidine moiety containing compound.

In one aspect, In one embodiment; the hair straightening adjuvant can be selected from one or more guanidine moiety containing compounds selected from guanidine; guanidine hydrochloride; guanidine acetate; guanidine sulfate; guanidine carbonate; guanidine bicarbonate; guanidine nitrate; guanidine phosphate; guanidine sulfamate; aminoguanidine; aminoguanidine hydrochloride; aminoguanidine nitrate; aminoguanidine sulfate; aminoguanidine bicarbonate; 1,3-diaminoguanidine hydrochloride; cyanoguanidine; nitroguanidine; 1-methyl-3-nitroguanidine; 1-acetylguanidine; chloroacetylguanidine hydrochloride; guanidine acetic acid guanylurea; guanylurea phosphate; phenylguanidine carbonate; phenylguanidine bicarbonate; 1,3-diphenylguanidine; 1,3-di-o-tolylguanidine; methyl 4-guanidino-2-methoxybenzoate hydrochloride; 1-methylguanidine hydrochloride; 1,1-dimethylguanidine hydrochloride; 1,1-dimethylguanidine sulfate; 1-ethylguanidine hydrochloride; 1-ethylguanidine sulfate; 1-octylguanidine hemisulfate; 1,1-diethylguanidine hydrochloride; N-tert-butoxycarbonylguanidine (N-Boc guanidine) 1,3-bis(tert-butoxycarbonyl)guanidine (Di-Boc guanidine); carbamic acid; N,N′-[[(1,1-dimethylethoxy)carbonyl]carbonimidoyl]bis-; C,C′-bis(1,1-dimethylethyl) ester (N,N′,N″-Tri-Boc guanidine); creatine; creatine monohydrate; creatinine; creatinine hydrochloride; creatine ethyl ester; agmatine; agmatine sulfate; guanidineacetic acid; guanidinosuccinic acid; 3-guanidinopropionic acid; 4-guanidinobutyric acid; 5-guanidinovaleric acid; 4-guanidine benzoic acid; 4-guanidine benzoic acid hydrochloride; 4-guanidino-2-methoxybenzoic acid; beta-N-methylguanidinopropionic acid; N-methylguanidinopropionic acid; N-(2-hydroxyethyl)guanidine; N-(3-hydroxypropyl)guanidine; biguanide hydrochloride; N-methylbiguanide hydrochloride; N-ethylbiguanide hydrochloride; N-propylbiguanide hydrochloride; N-butylbiguanide hydrochloride; 1-o-tolylbiguanide; 1,1-dimethylbiguanide hydrochloride; 1-(3-chloro-4-fluorophenyl)biguanide hydrochloride; 1-(2-chlorophenyl)biguanide hydrochloride; 1-(4-chlorophenyl)biguanide hydrochloride; 1-(2-fluorophenyl)biguanide hydrochloride; 1-(3-fluorophenyl)biguanide hydrochloride; 1-(4-fluorophenyl)biguanide hydrochloride; 1-(2,3-dichlorophenyl)biguanide hydrochloride; 1-(2,4-dichlorophenyl)biguanide hydrochloride; 1-(2,5-dichlorophenyl)biguanide hydrochloride; 1-(2,6-dichlorophenyl)biguanide hydrochloride; 1-(3,4-dichlorophenyl)biguanide hydrochloride; 1-(3,5-dichlorophenyl)biguanide hydrochloride; 1-(2,4-difluorophenyl)biguanide hydrochloride; 1-(2,5-difluorophenyl)biguanide hydrochloride; 1-[4-(trifluoromethyl)phenyl]biguanide hydrochloride; 1-[3,5-di-(trifluoromethyl)phenyl]biguanide hydrochloride; 1-[4-(trifluoromethoxy)phenyl]biguanide hydrochloride; 1-phenylbiguanide; 1-phenylbiguanide carbonate; 1,1′-hexamethylenebis[5-(4-chlorophenyl)biguanide]; 1,1′-hexamethylenebis(5-[p-chlorophenyl]biguanide) dihydrochloride; [({[Amino(imino)methyl]amino}carbothioyl)amino]benzene (Aldrich® CDS000226); N-cyclohexyl guanidine hydrochloride; N-benzylguanidine hydrochloride; N-benzyl-N-methylguanidine hemisulfate; 1,1,3,3-tetramethylguanidine; 1,1,3,3-tetramethylguanidine hydrochloride; 2-tert-butyl-1,1,3,3-tetramethylguanidine; 2-tert-butyl-1,1,3,3-tetramethylguanidine hydrochloride; 4-morpholinecarboximidoylguanidine hydrochloride; L-arginine; D-arginine; DL-arginine; arginic acid; N-tosyl-L-Arginine; N-amidino-N-(2,3-dihydroxypropyl)glycine; N-amidinotaurine; 2-imino-1-imidazolidineacetic acid; 1-(2,2-diethoxyethyl)guanidine; (2S)-3-carbamimidamido-2-{[(9H-fluoren-9-ylmethoxy)carbonyl]amino}propanoic acid (Fmoc-L-2-amino-3-guanidinopropionic acid; Aldrich® CDS019428); 1,8-bis(tetramethylguanidino)naphthalene; 1H-pyrazole-1-carboxamidine hydrochloride; 5-hydroxy-3-methyl-1H-pyrazole-1-carboximidamide; 3,5-diamino-1H-1,2,4-triazole-1-carboximidamide hydrochloride; 2-guanidone-4-thiazolidone; 2-guanidinobenzimidazole; 2-guanidinobenzoxazole; 2-guanidinobenzothiazole; pyrrolidinoformamidine hydrochloride; 2,3,4,6,7,8,9,10-octahydropyrimido[1,2-a][1,3]diazepine (Htbu); 2,5,6,7,8,9-hexahydro-3H-imidazo[1,2-a][1,3]diazepine (Htbd); 1,5,7-triazabicyclo[4.4.0]dec-5-ene (Hhpp); 3,3,9,9-tetramethyl-1,2,7-triazabicyclo[4.4.0]dec-5-ene (HTMhpp); 3,3,9,9-tetraethyl-1,2,7-triazabicyclo[4.4.0]dec-5-ene (HTEhpp); 1,5,7-triazabicyclo[4.3.0]non-6-ene (Htbn); 1,4,6-triazabicyclo[3.3.0]oct-4-ene (Htbo); and mixtures thereof.

It has been discovered that the combination of the at least one heterocyclic compound containing two heteroatoms at least one of said heteroatoms being oxygen and the other being selected from nitrogen and oxygen in a 5 to 8-membered ring with the at least one guanidine moiety containing compound provides a hair straightening formulation with synergistic efficacy when compared to hair straightening formulations utilizing the said heterocyclic compound alone. In one embodiment, hair straightening formulations which employ a cyclic alkylene carbonate of Formula (IB) (e.g., ethylene carbonate, propylene carbonate and mixtures thereof) in combination with a guanidine moiety containing compound (e.g., guanidine, guanidine acetic acid, L-arginine, creatinine, creatinine ethyl ester, 1,5,7-triazabicyclo[4.4.0]dec-5-ene (TBD), and their salts, and mixtures thereof) exhibit synergy and enhanced efficacy.

In one embodiment, the composition (and treatment process described herein) is free (0 wt. %) or substantially free (less than 1 wt. %, such as less than 0.1 wt. %, or less than 0.01 wt. %, or less than 0.001 wt. %, based on the total weight of the composition) of thermally-activated agents/cross-linking agents other than those of Formulas (I) and (II). In one exemplary embodiment, the thermally-activated agent(s) of Formulas (I) and (II) are the sole thermally-activated hair straightening agents present in the hair straightening composition of the disclosed technology. In particular, the hair straightening composition and method are free (0 wt. %) or substantially free (less than 1 wt. %, such as less than 0.1 wt. %, or less than 0.01 wt. %, or less than 0.001 wt. %, based on the total weight of the composition) of formaldehyde or formaldehyde precursors. The exemplary method and composition of the disclosed technology use no formaldehyde either directly, in the composition, or through reaction, during the method, of any sources of formaldehyde. Similarly, the composition and process may be free (0 wt. %) or substantially free (less than 1 wt. %, or less than 0.1 wt. %, or less than 0.01 wt. %, or less than 0.001 wt. %, based on the total weight of the composition) of glyoxylic acid, urea, and derivatives thereof, which can break down to form irritants.

In one embodiment, the composition and treatment process are free (0 wt. %) or substantially free (less than 1 wt. %, or less than 0.1 wt. %, or less than 0.01 wt. %, or less than 0.001, based on the total weight of the composition) of cyclic mercapto (thiol) group-containing compounds. Cyclic mercapto group-containing compounds are cyclic compounds containing an S—H group linked directly to a ring carbon atom. In one embodiment the composition (and treatment method described herein) is free or substantially free (less than 1 wt. %, or less than 0.1 wt. %, or less than 0.01 wt. %, or less than 0.001 wt. %, based on the total weight of the composition) of all compounds which contain thiol groups, including aliphatic and cyclic thiol group-containing compounds.

In one embodiment, the composition and treatment process are free or substantially free (less than 1 wt. %, or less than 0.1 wt. %, or less than 0.01 wt. %, or less than 0.001 wt. %, based on the total weight of the composition) of sulfites which can break the bisulfide bond in the keratin fibers (that are typically used in permanent waving compositions).

In one embodiment, the hair straightening agent (a) and guanidine moiety containing adjuvant (b) of the present technology can be utilized in combination with any suitable thermally-activated hair straightening agent known in the art, including, formaldehyde; glyoxyloyl; carbocysteine; glyoxyl; malondialdehyde; succindialdehyde; glutaraldehyde; carboxylic acids selected from glyoxylic acid, gallic acid, tartaric acid, malic acid, maleic acid, itaconic acid, oxalic acid, malonic acid, mesoxalic acid, fumaric, succinic acid, iminodiacetic acid, galactartic acid, adipic acid, glutaric acid; citric acid, isocitric acid, 1,2,3,4-butanetetracarboxylic acid, and salts thereof; urea derivatives selected from urea, dimethylolethyleneurea, dimethylol dihydroxyethyleneurea, alkylated dimethylol dihydroxy ethylene urea; and combinations thereof.

In one embodiment, the hair straightening agent (a) and guanidine moiety containing adjuvant (b) of the present technology can be spiked into existing commercial hair straightening products to enhance the hair straightening efficacy thereof.

### Cosmetically Acceptable Excipients

As previously discussed a cosmetically acceptable excipient is a non-toxic, non-irritating substance used as a diluent, carrier vehicle, and/or an auxiliary additive for formulating components (a) and (b), which when mixed with the thermally-activated hair straightening agent(s) and adjuvant(s) of the disclosed technology provides a composition that is more suitably applied to the hair and/or imparts other desirable effects to the hair and/or scalp.

Cosmetically acceptable excipients used in the hair care industry can be broken down into several categories. Components from a category may be included or excluded from the final hair care composition depending on the use and desired form of the final composition (e.g., liquid, cream, mousse, gel, spray, lotion, pomade, and the like). The choice and amount of each optional excipient will vary with the purpose and character of the end product, and can be readily determined by one skilled in the formulation art and from the literature. It is recognized that excipients additives, adjuvants, and benefit agents set forth herein can serve more than one function in a composition, such as, for example, surfactants, emulsifiers, solubilizers, conditioners, emollients, humectants, lubricants, pH adjusting agents, and acid based preservatives. The categories of excipients include, but are not limited to the following.

### Solvent/Diluent/Carrier

In addition to the one or more thermally-activated agents and adjuvants present, the composition may include a solvent, diluent, and or carrier in which the thermally-activated agent(s) an adjuvant(s) is soluble or dispersible. Specific examples include water and/or ethanol.

The composition can be prepared as water-free or water-based formulations, and formulations containing water-miscible auxiliary solvents, diluents and/or carriers are also contemplated.

Useful solvents commonly employed are typically liquids, such as water (deionized, distilled or purified), alcohols, such as C₁-C₁₀ aliphatic and aromatic alcohols (including diols and triols, such as glycols, e.g., ethylene glycol, propylene glycol, and glycerin), polyols, and the like, and mixtures thereof.

Examples of solvents, other than water, include linear and branched alcohols, such as ethanol, propanol, isopropanol, hexanol, and the like; and aromatic alcohols, such as benzyl alcohol, cyclohexanol, and the like. Non-limiting examples of polyols include polyhydroxy alcohols, such as glycerin, polyethylene glycols, propylene glycol, butylene glycol, hexylene glycol, C₂ to C₄ alkoxylated alcohols and C₂ to C₄ alkoxylated polyols, such as ethoxylated, propoxylated, and butoxylated ethers of alcohols, diols, and polyols having about 2 to about 30 carbon atoms and 1 to about 40 alkoxy units, polypropylene glycol, polybutylene glycol, and the like. Other examples of non-aqueous solvents or diluents include silicones, and silicone derivatives, such as cyclomethicone, and the like, ketones such as acetone and methylethyl ketone; ethers such as dimethyl ether, dimethoxymethane and the like; natural and synthetic oils and waxes, such as vegetable oils, plant oils, animal oils, essential oils, Paraffinum Liquidum (mineral oil), C₇ to C₄₀ isoparaffins, alkyl carboxylic esters, such as ethyl acetate, amyl acetate, ethyl lactate, and the like, jojoba oil, shark liver oil, and the like. Some of the foregoing non-aqueous auxiliary solvents or diluents may also serve as conditioners and emulsifiers. For purposes of computing a weight basis in the composition, however, all of the liquids listed in this section are considered as solvents/diluents.

### Surfactants

The composition can also include one or more surfactants, such as anionic, cationic, amphoteric, and nonionic surfactants, as well as mixtures thereof.

The anionic surfactant can be any of the anionic surfactants known or previously used in the art of aqueous surfactant compositions. Suitable anionic surfactants include but are not limited to alkyl sulfates, alkyl ether sulfates, alkyl sulphonates, alkaryl sulfonates, α-olefin-sulphonates, alkylamide sulphonates, alkarylpolyether sulphates, alkylamidoether sulphates, alkyl monoglyceryl ether sulfates, alkyl monoglyceride sulfates, alkyl monoglyceride sulfonates, alkyl succinates, alkyl sulfosuccinates, alkyl sulfosuccinamates, alkyl ether sulphosuccinates, alkyl amidosulfosuccinates; alkyl sulphoacetates, alkyl phosphates, alkyl ether phosphates, alkyl ether carboxylates, alkyl amidoethercarboxylates, N-alkylamino acids, N-acyl amino acids, alkyl peptides, N-acyl taurates, alkyl isethionates, carboxylate salts wherein the acyl group is derived from fatty acids; and the alkali metal, alkaline earth metal, ammonium, amine, and triethanolamine salts thereof.

In one aspect, the cation moiety of the forgoing salts is selected from sodium, potassium, magnesium, ammonium, mono-, di- and triethanolamine salts, and mono-, di-, and tri-isopropylamine salts. The alkyl and acyl groups of the foregoing surfactants contain from about 6 to about 24 carbon atoms in one aspect, from 8 to 22 carbon atoms in another aspect and from about 12 to 18 carbon atoms in a further aspect and may be unsaturated. The aryl groups in the surfactants are selected from phenyl or benzyl. The ether containing surfactants set forth above can contain from 1 to 10 ethylene oxide and/or propylene oxide units per surfactant molecule in one aspect, and from 1 to 3 ethylene oxide units per surfactant molecule in another aspect.

Examples of suitable anionic surfactants include, but are not limited to, sodium, potassium, lithium, magnesium, and ammonium salts of laureth sulfate, trideceth sulfate, myreth sulfate, C₁₂-C₁₃ pareth sulfate, C₁₂-014 pareth sulfate, and C₁₂-C₁₅ pareth sulfate, ethoxylated with 1, 2, and 3 moles of ethylene oxide; sodium, potassium, lithium, magnesium, ammonium, and triethanolamine lauryl sulfate, coco sulfate, tridecyl sulfate, myrstyl sulfate, cetyl sulfate, cetearyl sulfate, stearyl sulfate, oleyl sulfate, and tallow sulfate, disodium lauryl sulfosuccinate, disodium laureth sulfosuccinate, sodium cocoyl isethionate, sodium C₁₂-C₁₄ olefin sulfonate, sodium laureth-6 carboxylate, sodium methyl cocoyl taurate, sodium cocoyl glycinate, sodium myristyl sarcocinate, sodium dodecylbenzene sulfonate, sodium cocoyl sarcosinate, sodium cocoyl glutamate, potassium myristoyl glutamate, triethanolamine monolauryl phosphate, and fatty acid soaps, including the sodium, potassium, ammonium, and triethanolamine salts of a saturated and unsaturated fatty acids containing from about 8 to about 22 carbon atoms.

The cationic surfactants may act as conditioning agents and assist in the heating step by ensuring that the heating device runs smoothly over the hair fibers. While the surfactants may also help to increase viscosity, they are not considered as rheology modifiers for purposes of describing the exemplary embodiment herein.

The cationic surfactants can be any of the cationic surfactants known or previously used in the art of aqueous surfactant compositions. Suitable classes of cationic surfactants include, but are not limited to, alkyl amines, alkyl imidazolines, ethoxylated amines, quaternary compounds, and quaternized esters. In addition, alkyl amine oxides can function as a cationic surfactant at a low pH.

Alkylamine surfactants can be salts of primary, secondary and tertiary fatty C₁₀-C₃₂ alkylamines, substituted or unsubstituted, and substances sometimes referred to as “amidoamines”. Examples of alkylamines and salts thereof include dimethyl cocamine, dimethyl palmitamine, dioctylamine, dimethyl stearamine, dimethyl soyamine, soyamine, myristyl amine, tridecyl amine, ethyl stearylamine, N-tallowpropane diamine, ethoxylated stearylamine, dihydroxy ethyl stearylamine, arachidylbehenylamine, dimethyl lauramine, stearylamine hydrochloride, soyamine chloride, stearylamine formate, N-tallowpropane diamine dichloride, and amodimethicone (INCI name for a silicone polymer and blocked with amino functional groups, such as aminoethylamino propylsiloxane).

Examples of amidoamines and salts thereof include stearamido propyl dimethyl amine, stearamidopropyl dimethylamine citrate, palmitamidopropyl diethylamine, and cocamidopropyl dimethylamine lactate.

Examples of alkyl imidazoline surfactants include alkyl hydroxyethyl imidazoline, such as stearyl hydroxyethyl imidazoline, coco hydroxyethyl imidazoline, ethyl hydroxymethyl oleyl oxazoline, and the like.

Examples of ethyoxylated amines include PEG-cocopolyamine, PEG-15 tallow amine, quaternium-52, and the like.

Among the quaternary ammonium compounds useful as cationic surfactants, some correspond to the general formula: (R¹⁵R¹⁶R¹⁷R¹⁸N⁺)E⁻, wherein R¹⁵, R¹⁶, R¹⁷, and R¹⁸ are independently selected from an aliphatic group having from 1 to about 22 carbon atoms, or an aromatic, alkoxy, polyoxyalkylene, alkylamido, hydroxyalkyl, aryl or alkylaryl group having 1 to about 22 carbon atoms in the alkyl chain; and E⁻is a salt-forming anion such as those selected from halogen, (e.g., chloride, bromide), acetate, citrate, lactate, glycolate, phosphate, nitrate, sulfate, and alkylsulfate. The aliphatic groups can contain, in addition to carbon and hydrogen atoms, ether linkages, ester linkages, and other groups such as amino groups. The longer chain aliphatic groups, e.g., those of about 12 carbons, or higher (C₁₀-C₃₂ in the alkyl chain), can be saturated or unsaturated. In one aspect, the aryl groups are selected from phenyl and benzyl.

Exemplary quaternary ammonium surfactants include, but are not limited to cetyl trimethylammonium chloride (cetrimonium chloride), cetylpyridinium chloride, dicetyl dimethyl ammonium chloride, dihexadecyl dimethyl ammonium chloride, stearyl dimethyl benzyl ammonium chloride, dioctadecyl dimethyl ammonium chloride, dieicosyl dimethyl ammonium chloride, didocosyl dimethyl ammonium chloride, dihexadecyl dimethyl ammonium chloride, dihexadecyl dimethyl ammonium acetate, behenyl trimethyl ammonium chloride (behentrimonium chloride), benzalkonium chloride, benzethonium chloride, and di(coconutalkyl) dimethyl ammonium chloride, ditallowdimethyl ammonium chloride, di(hydrogenated tallow) dimethyl ammonium chloride, di(hydrogenated tallow) dimethyl ammonium acetate, ditallowdimethyl ammonium methyl sulfate, ditallow dipropyl ammonium phosphate, and ditallow dimethyl ammonium nitrate.

At low pH, amine oxides can protonate and behave similarly to N-alkyl amines. Examples include, but are not limited to, dimethyl-dodecylamine oxide, oleyldi(2-hydroxyethyl) amine oxide, dimethyltetradecylamine oxide, di(2-hydroxyethyl)-tetradecylamine oxide, dimethylhexadecylamine oxide, behenamine oxide, cocamine oxide, decyltetradecylamine oxide, dihydroxyethyl C₁₂₋₁₅ alkoxypropylamine oxide, dihydroxyethyl cocamine oxide, dihydroxyethyl lauramine oxide, dihydroxyethyl stearamine oxide, dihydroxyethyl tallowamine oxide, hydrogenated palm kernel amine oxide, hydrogenated tallowamine oxide, hydroxyethyl hydroxypropyl C₁₂-C₁₅ alkoxypropylamine oxide, lauramine oxide, myristamine oxide, cetylamine oxide, oleamidopropylamine oxide, oleamine oxide, palmitamine oxide, PEG-3 lauramine oxide, dimethyl lauramine oxide, potassium trisphosphonomethylamine oxide, soyamidopropylamine oxide, cocamidopropylamine oxide, stearamine oxide, tallowamine oxide, and mixtures thereof.

Particularly useful cationic surfactants include, but are not limited to, fatty acid derivatives, such as fatty (C₁₀-C₃₂ in the alkyl chain) alkylammonium chlorides, such as cetrimonium chloride and behentrimonium chloride. Such surfactants may also serve as conditioning agents and emollients.

Amphoteric or zwitterionic surfactants are molecules that contain acidic and basic moieties and have the capacity of behaving either as an acid or a base. Suitable surfactants can be any of the amphoteric surfactants known or previously used in the art of aqueous surfactant compositions. Exemplary amphoteric surfactant classes include but are not limited to amino acids (e.g., N-alkyl amino acids and N-acyl amino acids), betaines, sultaines, and alkyl amphocarboxylates.

Amino acid based surfactants suitable herein include surfactants represented by the formula:

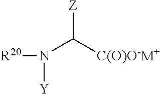

where:
R²⁰ represents a saturated or unsaturated hydrocarbon group having 10 to 22 carbon atoms or an acyl group containing a saturated or unsaturated hydrocarbon group having 9 to 22 carbon atoms, Y is hydrogen or methyl, Z is selected from hydrogen, —CH₃, —CH(CH₃)₂, —CH₂CH(CH₃)₂, —CH(CH₃)CH₂CH₃, —CH₂C₆H₅, —CH₂C₆H₄OH, —CH₂OH, —CH(OH)CH₃, —(CH₂)₄NH₂, —(CH₂)₃NHC(NH)NH₂, —CH₂C(O)O⁻M⁺, —(CH₂)₂C(O)O⁻M⁺. M is a salt forming cation. In one aspect, R²⁰ represents a radical selected from a linear or branched C₁₀ to C₂₂ alkyl group, a linear or branched C₁₀ to 022 alkenyl group, an acyl group represented by R²¹C(O)—, wherein R²¹ is selected from a linear or branched C₉ to C₂₂ alkyl group, a linear or branched C₉ to C₂₂ alkenyl group. In one aspect, M⁺ is selected from sodium, potassium, ammonium, and triethanolamine (TEA).

The amino acid surfactants can be derived from the alkylation and acylation of α-amino acids such as, for example, alanine, arginine, aspartic acid, glutamic acid, glycine, isoleucine, leucine, lysine, phenylalanine, serine, tyrosine, and valine. Representative N-acyl amino acid surfactants are, but not limited to the mono- and di-carboxylate salts (e.g., sodium, potassium, ammonium and TEA) of N-acylated glutamic acid, for example, sodium cocoyl glutamate, sodium lauroyl glutamate, sodium myristoyl glutamate, sodium palmitoyl glutamate, sodium stearoyl glutamate, disodium cocoyl glutamate, disodium stearoyl glutamate, potassium cocoyl glutamate, potassium lauroyl glutamate, and potassium myristoyl glutamate; the carboxylate salts (e.g., sodium, potassium, ammonium and TEA) of N-acylated alanine, for example, sodium cocoyl alaninate, and TEA lauroyl alaninate; the carboxylate salts (e.g., sodium, potassium, ammonium and TEA) of N-acylated glycine, for example, sodium cocoyl glycinate, and potassium cocoyl glycinate; the carboxylate salts (e.g., sodium, potassium, ammonium and TEA) of N-acylated sarcosine, for example, sodium lauroyl sarcosinate, sodium cocoyl sarcosinate, sodium myristoyl sarcosinate, sodium oleoyl sarcosinate, and ammonium lauroyl sarcosinate; and mixtures of the foregoing surfactants.

The betaines and sultaines useful in the composition are selected from alkyl betaines, alkylamino betaines, and alkylamido betaines, as well as the corresponding sulfobetaines (sultaines) represented by the formulas:

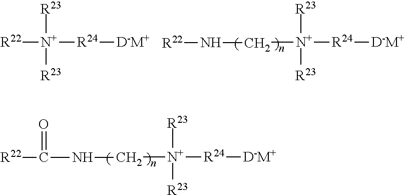

where:
R²² is a C₇-C₂₂ alkyl or alkenyl group, each R²³ independently is a C₁-C₄ alkyl group, R²⁴ is a C₁-C₅ alkylene group or a hydroxy substituted C₁-C₅ alkylene group, n is an integer from 2 to 6, D is a carboxylate or sulfonate group, and M is a salt forming cation. In one aspect, R²² is a C₁₁-C₁₈ alkyl group or a C₁₁-C₁₈ alkenyl group. In one aspect, R²³ is methyl. In one aspect, R²⁴ is methylene, ethylene or hydroxy propylene. In one aspect, n is 3. In a further aspect, M is selected from sodium, potassium, magnesium, ammonium, and mono-, di- and triethanolamine cations.

Examples of suitable betaines include, but are not limited to, lauryl betaine, coco betaine, oleyl betaine, cocohexadecyl dimethylbetaine, lauryl amidopropyl betaine, cocoamidopropyl betaine, and cocamidopropyl hydroxysultaine.

The alkylamphocarboxylates, such as the alkylamphoacetates and alkylamphopropionates (mono- and disubstituted carboxylates), can be represented by the formula:

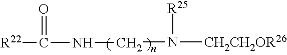

where:
R²² is a C₇-C₂₂ alkyl or alkenyl group, R²⁵ is —CH₂C(O)O⁻M⁺, —CH₂CH₂C(O)O⁻M⁺, or —CH₂CH(OH)CH₂SO₃⁻M⁺, R²⁶ is a hydrogen or —CH₂C(O)O⁻M⁺, and M is a cation selected from sodium, potassium, magnesium, ammonium, and mono-, di- and triethanolamine.

Exemplary alkylamphocarboxylates include, but are not limited to, sodium cocoamphoacetate, sodium lauroamphoacetate, sodium capryloamphoacetate, disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, and disodium capryloamphodipropionate.

The nonionic surfactant can be any of the nonionic surfactants known or previously used in the art of aqueous surfactant compositions. Suitable nonionic surfactants include, but are not limited to, aliphatic (C₆-C₁₈) primary or secondary linear or branched chain acids, alcohols or phenols; alkyl ethoxylates; alkyl phenol alkoxylates (especially ethoxylates and mixed ethoxy/propoxy moieties); block alkylene oxide condensates of alkyl phenols; alkylene oxide condensates of alkanols; and ethylene oxide/propylene oxide block copolymers. Other suitable nonionic surfactants include mono- or dialkyl alkanolamides; alkyl polyglucosides (APGs); sorbitan fatty acid esters; polyoxyethylene sorbitan fatty acid esters; polyoxyethylene sorbitol esters; polyoxyethylene acids, and polyoxyethylene alcohols. Other examples of suitable nonionic surfactants include coco mono- or diethanolamide, coco glucoside, decyl diglucoside, lauryl diglucoside, coco diglucoside, polysorbate 20, 40, 60, and 80, ethoxylated linear alcohols, cetearyl alcohol, lanolin alcohol, stearic acid, glyceryl stearate, PEG-100 stearate, laureth 7, and oleth 20.

The nonionic surfactants also include alkoxylated methyl glucosides such as, for example, methyl gluceth-10, methyl gluceth-20, PPG-10 methyl glucose ether, and PPG-20 methyl glucose ether, available from Lubrizol Advanced Materials, Inc., under the trade names, Glucam® E10, Glucam® E20, Glucam® P10, and Glucam® P20, respectively; and hydrophobically modified alkoxylated methyl glucosides, such as PEG 120 methyl glucose dioleate, PEG-120 methyl glucose trioleate, and available from Lubrizol Advanced Materials, Inc., under the trade names, Glucamate® DOE-120 and Glucamate™ LT, respectively, are also suitable. Other exemplary hydrophobically modified alkoxylated methyl glucosides are disclosed in U.S. Pat. Nos. 6,573,375 and 6,727,357.

Other nonionic surfactants which can be utilized in the composition are set forth in more detail in WO 99/21530, U.S. Pat. Nos. 3,929,678, 4,565,647, 5,720,964, and 5,858,948. In addition, suitable surfactants are also described in McCutcheon's Emulsifiers and Detergents (North American and International Editions, by Schwartz, Perry and Berch).

While the amounts of the surfactant utilized in a composition comprising the exemplary thermally-activated agent can vary widely depending on a desired formulation, the amounts which are often utilized generally range from 1 wt. % to 80 wt. %, on an actives basis. For example, the surfactant may be present in the composition, on an actives basis, at a total concentration of 0.001-20 wt. %, e.g., at least 0.1 wt. %.

### Emulsifiers

Useful emulsifiers include, but are not limited to, fatty alcohols and fatty acids, as well as their alkoxylates; partial esters of polyols having 2 to 6 carbon atoms, e.g., partial esters of polyglycerols; naturally occurring gums, e.g., gum acacia or gum tragacanth; naturally occurring phosphatides, e.g. soybean lecithin; sorbitan monooleate derivatives; wool fats; wool alcohols; sorbitan esters; monoglycerides; and emulisifying silicones, organosiloxanes, emulsifying waxes, and combinations thereof. The fatty alcohols and fatty acids may be obtained from natural sources and thus be a mixture of alcohols and acids, respectively.

Useful fatty alcohols include, but are not limited to, C₁₂-C₃₂ or C₁₂-C₂₂ fatty alcohols, alkoxylated alcohols, such as C₁₂-C₃₂ or C₁₂-C₂₂ alkoxylated alcohols.

Useful fatty acids include, but are not limited to, C₁₂-C₃₂ or C₁₂-C₂₂ fatty acids, alkoxylated fatty acids, such as C₁₂-C₃₂ or C₁₂-C₂₂ alkoxylated fatty acids.

In one aspect, the fatty alcohol and fatty acid alkoxylates each have 2 to 150 units of ethylene oxide, propylene oxide, and combinations of ethylene oxide/propylene oxide present in the molecule in one aspect and 10 to 80 in another aspect.

One example of a fatty alcohol useful herein is cetearyl alcohol, which is a mixture of fatty alcohols, predominantly cetyl and stearyl alcohols. Such fatty alcohols may also serve as emollients and/or conditioning agents.

Suitable emulsifiers also include fatty alcohol polyoxyethylene ethers, such as ceteareth-n (where n represents the number of polyethylene units in the chain, such as from 2-100, e.g., 5-50), such as ceteareth-20.

The emulsifiers based on the esters and partial esters of polyols having 2 to 6 carbon atoms can be condensed with linear saturated and unsaturated fatty acids having 12 to 30 carbon atoms. For example, monoesters and diesters of glycerol or ethylene glycol or the monoesters of propylene glycol are formed from the condensation of glycerol, ethylene glycol, or propylene glycol with saturated and unsaturated/or C₁₂-C₃₀ fatty acids, e.g., glyceryl stearate and glycol stearate.

Suitable emulsifiers also include methyl glucosides, C₈-C₂₂ alkyl glucosides and C₈-C₂₂ alkyl polyglucosides (APGs). Exemplary alkyl glucosides and alkyl polyglycosides can be selected from octyl glucoside, decyl glucoside, lauryl glucoside, palmityl glucoside, isostearyl glucoside, stearyl glucoside, arachidyl glucoside and behenyl glucoside, as well as the oligomers thereof, and mixtures of alkyl glucosides and alkyl polyglucosides. Exemplary methyl glucosides include, methyl glucose sesquistearate, methyl glucose dioleate, and PEG-20 methyl glucose sesquistearate, sold under the tradenames Glucate™ SS, Glucate™ SSE-20, and Glucate DO emulsifiers, respectively, by Lubrizol Advanced Materials, Inc.

The emulsifier may be suitably present at 0.01-20 wt. % of the composition, such as at least 0.1 wt. %, e.g., up to 5 wt. %, which for purposes of computing amounts present in the composition, excludes those listed above as rheology modifiers.

### Humectants

Humectants are defined as materials that absorb or release water vapor, depending on the relative humidity of the environment, (Harry's Cosmeticology, Chemical Publishing Company Inc., 1982 p. 266). Suitable humectants that include, but are not limited to, allantoin; pyrrolidonecarboxylic acid and its salts; hyaluronic acid and salts thereof; sorbic acid and salts thereof; urea, lysine, cystine, and amino acids other than guanidine moiety containing amino acids; polyhydroxy alcohols such as glycerin, propylene glycol, hexylene glycol, hexanetriol, ethoxydiglycol, dimethicone copolyol, and sorbitol, and the esters thereof; polyethylene glycol; glycolic acid and glycolate salts (e.g. ammonium and quaternary alkyl ammonium); chitosan; aloe-vera extracts; algae extract; honey and derivatives thereof; inositol; lactic acid and lactate salts (e.g. ammonium and quaternary alkyl ammonium); sugars and starches; sugar and starch derivatives (e.g., glucose alkoxylated glucose, mannitol, xyliyol); DL-panthenol; magnesium ascorbyl phosphate, arbutin, kojic acid, lactamide monoethanolamine; acetamide monoethanolamine; and the like, and mixtures thereof. Humectants also include the C₃ to C₆ diols and triols, such as glycerin, propylene glycol, butane-1,2,3-triol, hexylene glycol, hexanetriol, and the like, and mixtures thereof. Ethoxylated methyl glucose ethers containing an average of 5 to 30 moles of ethoxylation, such as, for example, those available under the NCI names Lauryl Methyl Gluceth-10 Hydroxypropyldimonium chloride, Methyl Gluceth-10 and Methyl Gluceth-20, are suitable,

Such humectants may be present at from 0.01-20 wt. % of the composition, such as at least 0.1 wt. %, or at least 1 wt. %, e.g., up to 8 wt. %, or up to 5 wt. %.

### Emollients

An emollient is defined as a substance which regulates the rate and quantity of water uptake by the skin (Handbook of Cosmetic Science and Technology, Elsevier Science Publishing, 1993, p. 175). Suitable emollients include mineral oil; vegetable oil; hydrogenated vegetable oil, stearic acid; fatty alcohols such as cetyl alcohol, cetearyl alcohol, myristyl alcohol, behenyl alcohol, and lauryl alcohol, cetyl acetate in acetylated lanolin alcohol, benzoate esters such as 012 to 015 alkyl benzoates, isostearyl benzoate, dicaprylyl maleate, petrolatum, lanolin, coco butter, shea butter, beeswax and esters thereof, ethoxylated fatty alcohol esters such as ceteareth-20, oleth-5, and ceteth-5, alkoxylated fatty acid esters such as polyethylene glycol 400 propoxylated monolaurate, avocado oil or glycerides, sesame oil or glycerides, safflower oil or glycerides, sunflower oil or glycerides, and other mono-, di-, and triglycerides of natural vegetable and botanical oils, such as, for example, caprylic triglyceride, capric triglyceride, caprylic/capric triglyceride, and caprylic/capric/lauric triglyceride, Guerbet esters such as G-20, G-36, G-38, and G-66 marketed by Lubrizol Advanced Materials, Inc., botanical seed oils, volatile silicone oils, non-volatile emollients, and the like; and mixtures thereof.

Suitable non-volatile emollients include fatty acid and fatty alcohol esters, highly branched hydrocarbons, and the like, and mixtures thereof. Such fatty acid and fatty alcohol esters include decyl oleate, butyl stearate, octyl stearate, myristyl myristate, isostearyl hydroxystearate, isostearyl isostearate, di isopropyl sebacate, octyldodecyl stearoylstearate, octylhydroxystearate, di-isopropyl adipate, isopropyl myristate, isopropyl palmitate, ethyl hexyl palmitate, isodecyl neopentanoate C₁₂ to C₁₅ alcohol benzoate, diethyl hexyl maleate, PPG-14 butyl ether and PPG-2 myristyl ether propionate, cetearyl octanoate, and the like, and mixtures thereof. Suitable highly branched hydrocarbons include isohexadecane and the like, and mixtures thereof.

Suitable volatile emollients include the volatile silicones, such as cyclic or linear polydimethylsiloxanes, and the like. The number of silicon atoms in cyclic silicones can range from about 3 to about 7 in one aspect of the disclosed technology, and in another aspect from 4 to 5. Exemplary volatile silicones, both cyclic and linear, are available from Dow Corning Corporation as Dow Corning 344, 345, and 200. The linear volatile silicones typically have viscosities of less than about 5 cP at 25° C., while the cyclic volatile silicones typically have viscosities of less than about 10 cP at 25° C. “Volatile” means that the silicone has a measurable vapor pressure. A description of volatile silicones can be found in Todd and Byers, “Volatile Silicone Fluids for Cosmetics”, Cosmetics and Toiletries, Vol. 91, January 1976, pp. 27-32.

Other emollient silicones include polydimethylsiloxane gums, aminosilicones, phenylsilicones, polydimethyl siloxane, polydiethylsiloxane, polymethylphenylsiloxane, polydimethylsiloxane gums, polyphenyl methyl siloxane gums, amodimethicone, trimethylsilylamodimethicone, diphenyl-dimethyl polysiloxane gums, and the like.

The emollients, if present (alone or in combination), range from about 1 wt. % to about 15 wt. % in one aspect, from about 2 wt. % to about 10 wt. % in another aspect, and from about 3 wt. % to about 5 wt. % in a further aspect, based of the total weight of the soap composition.

### Conditioning Agents

The thermally-activated hair straightening compositions of the disclosed technology also can include a suitable hair conditioning agent. The choice of hair conditioning agent is limited only by its ability to generate a substantive conditioned effect on the straightened hair without interfering with the efficacy of the active hair straightening components. Exemplary conditioning agents include, but are not limited to, silicones and silicone derivatives, cationic compounds and polymers, hydrocarbon oils, natural oils, natural and synthetic waxes, ester oils and combinations thereof.

### 1. Silicones and Silicone Derivatives

The silicone conditioning agent may comprise volatile silicones, non-volatile silicones, and mixtures thereof. If volatile silicones are present, they are typically employed as a solvent or carrier for commercially available forms of non-volatile silicone fluid conditioning agents such as oils and gums and resins. Volatile silicone fluids are often included in the conditioning package to improve silicone fluid deposition efficacy or to enhance the shine, sheen or glossiness of the hair. Volatile silicone materials are frequently included in formulations to enhance sensory attributes (e.g., feel) on the hair and scalp.

In one aspect, the silicone conditioning agent is non-volatile and includes silicone oils, gums, resins and mixtures thereof. By non-volatile is meant that the silicone has a very low vapor pressure at ambient temperature conditions (e.g., less than 2 mm Hg at 20° C.). The non-volatile silicone conditioning agent has a boiling point above about 250° C. in one aspect, above about 260° C. in another aspect, and above about 275° C. in a further aspect. Background information on silicones including sections discussing silicone oils, gums, and resins, as well as their manufacture, are found in Encyclopedia of Polymer Science and Engineering, vol. 15, 2d ed., pp 204-308, John Wiley & Sons, Inc. (1989).

### A. Silicone Oil

In one aspect, the silicone conditioning agent is silicone oil selected from a polyorganosiloxane material. In one aspect, the polyorganosiloxane material can be selected from polyalkylsiloxanes, polyarylsiloxanes, polyalkylarylsiloxanes, hydroxyl terminated polyalkylsiloxanes, polyarylalkylsiloxanes, amino functional polyalkylsiloxanes, quaternary functional polyalkylsiloxanes, and mixtures thereof.

In one aspect, the silicone oil conditioning agent includes polyorganosiloxanes represented by the formula:

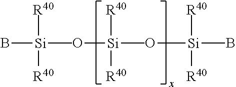

wherein B independently represents hydroxy, methyl, methoxy, ethoxy, propoxy, and phenoxy; R⁴⁰ independently represents methyl, ethyl, propyl, phenyl, methylphenyl, phenylmethyl, a primary, secondary or tertiary amine, a quaternary group selected from a group selected from:
—R⁴¹—N(R⁴²)CH₂CH₂N(R⁴²)₂;
—R⁴¹—N(R⁴²)₂;
—R⁴¹—N⁺(R⁴²)₃CA⁻; and
—R⁴¹—N(R⁴²)CH₂CH₂N⁺(R⁴²)H₂CA⁻;
   wherein R⁴¹ is a linear or branched, hydroxyl substituted or unsubstituted alkylene or alkylene ether moiety containing 2 to 10 carbon atoms; R⁴² is hydrogen, C₁-C₂₀ alkyl (e.g, methyl), phenyl or benzyl; q is an integer ranging from about 2 to about 8; CA⁻ is a halide ion selected from chlorine, bromine, iodine and fluorine; and x is an integer ranging from about 7 to about 8000 in one aspect, from about 50 to about 5000 in another aspect, form about 100 to about 3000 in still another aspect, and from about 200 to about 1000 in a further aspect.

In one aspect, the amino functional polyalkylsiloxane can be represented by the formula:

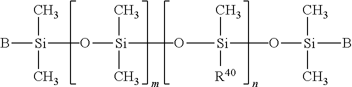

wherein B independently represents hydroxy, methyl, methoxy, ethoxy, propoxy, and phenoxy; and R⁴⁰ is selected from:
—R⁴¹—N(R⁴²)CH₂CH₂N(R⁴²)₂;
—R⁴¹—N(R⁴²)₂;
—R⁴¹—N⁺(R⁴²)₃CA⁻; and
—R⁴¹—N(R⁴²)CH₂CH₂N⁺(R⁴²)H₂CA⁻
   wherein R⁴¹ is a linear or branched, hydroxyl substituted or unsubstituted alkylene or alkylene ether moiety containing 2 to 10 carbon atoms; R⁴² is hydrogen, C₁-C₂₀ alkyl (e.g, methyl), phenyl or benzyl; CA⁻ is a halide ion selected from chlorine, bromine, iodine and fluorine; and the sum of m+n ranges from about 7 to about 1000 in one aspect, from about 50 to about 250 in another aspect, and from about 100 to about 200 in another aspect, subject to the proviso that m or n is not 0. In one aspect B is hydroxy and R⁴⁰ is
   —(CH₂)₃NH(CH₂)₃NH₂. In another aspect B is methyl and R⁴⁰ is —(CH₂)₃NH(CH₂)₃NH₂. In still another aspect B is methyl and R⁴⁰ is a quaternary ammonium moiety represented by —(CH₂)₃OCH₂CH(OH)CH₂N⁺(R⁴²)₃CA⁻; wherein R⁴² and CA⁻ are as previously defined.

The silicone oil conditioning agents can have a viscosity ranging from about above about 25 to about 1,000,000 mPa·s at 25° C. in one aspect, from about 100 to about 600,000 mPa·s in another aspect, and from about 1000 to about 100,000 mPa·s still another aspect, from about 2,000 to about 50,000 mPa·s in yet another aspect, and from about 4,000 to about 40,000 mPa·s in a further aspect. The viscosity is measured by means of a glass capillary viscometer as described by Dow Corning Corporate Test Method CTM004, dated Jul. 20, 1970. In one aspect the silicone oils have an average molecular weight below about 200,000 daltons. The average molecular weight can typically range from about 400 to about 199,000 daltons in one aspect, from about 500 to about 150,000 daltons in another aspect, from about 1,000 to about 100,000 daltons in still another aspect, from about 5,000 to about 65,000 daltons in a further aspect.

Exemplary silicone oil conditioning agents include, but are not limited to, polydimethylsiloxanes (dimethicones), polydiethylsiloxanes, polydimethyl siloxanes having terminal hydroxyl groups (dimethiconols), polymethylphenylsiloxanes, phenylmethylsiloxanes, amino functional polydimethylsiloxanes (amodimethicones), and mixtures thereof.

### B. Silicone Gum

Another silicone conditioning agent useful in the disclosed technology is a silicone gum. A silicone gum is a polyorganosiloxane material of the same general structure of the silicone oils set forth above wherein B independently represents hydroxy, methyl, methoxy, ethoxy, propoxy, and phenoxy; R⁴⁰ independently represents methyl, ethyl, propyl, phenyl, methylphenyl, phenylmethyl, and vinyl. Silicone gums have a viscosity measured at 25° C. of greater than 1,000,000 mPa·s. The viscosity can be measured by means of a glass capillary viscometer as described above for the silicone oils. In one aspect the silicone gums have an average molecular weight about 200,000 daltons and above. The molecular weight can typically range from about 200,000 to about 1,000,000 daltons. It is recognized that the silicone gums described herein can also have some overlap with the silicone oils described previously. This overlap is not intended as a limitation on any of these materials.

Suitable silicone gums for use in the silicone component of compositions of the disclosed technology are polydimethylsiloxanes (dimethicones), optionally having terminal end groups such as hydroxyl (dimethiconols), polymethylvinylsiloxane, polydiphenylsiloxane, and mixtures thereof.

### C. Silicone Resins

Silicone resins can be included as a silicone conditioning agent suitable for use in the compositions of the disclosed technology. These resins are crosslinked polysiloxanes. The crosslinking is introduced through the incorporation of trifunctional and tetrafunctional silanes with monofunctional and/or difunctional silanes during manufacture of the silicone resin. As is well understood in the art, the degree of crosslinking that is required in order to result in a silicone resin will vary according to the specific silane units incorporated into the silicone resin. In general, silicone materials which have a sufficient level of trifunctional and tetra-functional siloxane monomer units (and hence, a sufficient level of crosslinking) such that they form a rigid or hard film are considered to be silicone resins. The ratio of oxygen atoms to silicon atoms is indicative of the level of crosslinking in a particular silicone material. Silicone materials which have at least about 1.1 oxygen atoms per silicon atom will generally be silicone resins herein. In one aspect, the ratio of oxygen:silicon atoms is at least about 1.2:1.0. Silanes used in the manufacture of silicone resins include monomethyl-, dimethyl-, trimethyl-, monophenyl-, diphenyl-, methylphenyl-, monovinyl-, and methylvinyl-chlorosilanes, and terachlorosilane, with the methyl substituted silanes being most commonly utilized.

Silicone materials and silicone resins can be identified according to a shorthand nomenclature system known to those of ordinary skill in the art as “MDTQ” nomenclature. Under this naming system, the silicone is described according to the presence of various siloxane monomer units which make up the silicone. The “MDTQ” nomenclature system is described in the publication entitled “Silicones: Preparation, Properties and Performance”; Dow Corning Corporation, 2005, and in U.S. Pat. No. 6,200,554.

Exemplary silicone resins for use in the compositions of the disclosed technology include, but are not limited to MQ, MT, MTQ, MDT and MDTQ resins. In one aspect, methyl is the silicone resin substituent. In another aspect, the silicone resin is selected from a MQ resins, wherein the M:Q ratio is from about 0.5:1.0 to about 1.5:1.0 and the average molecular weight of the silicone resin is from about 1000 to about 10,000 daltons.

### D. Volatile Silicones

The optional volatile silicones referred to above include linear polydimethylsiloxanes and cyclic polydimethylsiloxanes (cyclomethicones), and mixtures thereof. Volatile linear polydimethylsiloxanes (dimethicones) typically contain about 2 to about 9 silicon atoms, alternating with oxygen atoms in a linear arrangement. Each silicon atom is also substituted with two alkyl groups (the terminal silicon atoms are substituted with three alkyl groups), such as, for example, methyl groups. The cyclomethicones typically contain about 3 to about 7 dimethyl substituted silicon atoms in one aspect and from about 3 to about 5 in another aspect, alternating with oxygen atoms, in a cyclic ring structure. The term “volatile” means that the silicone has a measurable vapor pressure, or a vapor pressure of at least 2 mm of Hg at 20° C. The volatile silicones have a viscosity of 25 mPa·s or less at 25° C. in one aspect, from about 0.65 about to about 10 mPa·s in another aspect, from about 1 to about 5 mPa·s in still another aspect, and from about 1.5 to about 3.5 mPa·s in a further aspect. A description of linear and cyclic volatile silicones is found in Todd and Byers, “Volatile Silicone Fluids for Cosmetics”, Cosmetics and Toiletries, Vol. 91 (1), pp. 27-32 (1976), and in Kasprzak, “Volatile Silicones”, Soap/Cosmetics/Chemical Specialities, pp. 40-43 (December 1986).

Exemplary volatile linear dimethicones include, but are not limited to, hexamethyldisiloxane, octamethyltrisiloxane, decamethyltetrasiloxane, dodecamethylpentasiloxane and blends thereof. Volatile linear dimethicones and dimethicone blends are commercially available from Dow Corning Corporation as Dow Corning 200® Fluid (e.g., product designations 0.65 CST, 1 CST, 1.5 CST, and 2 CST) and Dow Corning® 2-1184 Fluid.

Exemplary volatile cyclomethicones are D4 cyclomethicone (octamethylcyclotetrasiloxane), D5 cyclomethicone (decamethylcyclopentasiloxane), D6 cyclomethicone, and blends thereof (e.g., D4/D5 and D5/D6). Volatile cyclomethicones and cyclomethicone blends are commercially available from Momentive Performance Materials Inc. as SF1173, SF1202, SF1256, and SF1258 silicone fluids, and Dow Corning Corporation as Dow Corning® 244, 245, 246, 345, and 1401 silicone fluids. Blends of volatile cyclomethicones and volatile linear dimethicones also can be employed.

The amount of silicone conditioner(s) in the compositions of the present technology should be sufficient to provide the desired conditioning performance to the hair, and generally ranges from about 0.01 to about 20 wt. % in one aspect, from about 0.05 to about 15 wt. % in another aspect, from about 0.1% to about 10 wt. % in still another aspect, and from about 1 to about 5 wt. % in a further aspect, based on the total weight of the composition.

### 2. Cationic Compounds and Polymers

Cationic Compounds refer to non-polymeric and polymeric compounds containing at least one cationic moiety or at least one moiety that can be ionized to form a cationic moiety. Typically these cationic moieties are nitrogen containing groups such as quaternary ammonium or protonated amino groups. The cationic protonated amines can be primary, secondary, or tertiary amines. In one aspect, the cationic conditioning compounds include quaternary nitrogen containing non-polymeric and polymeric materials that well known in the art for hair conditioning. Cationic conditioning compounds include non-polymeric compounds containing one quaternary ammonium salt moiety and polymeric compounds (polymers) containing at least one quaternary ammonium salt moiety.

In one aspect, the quaternary ammonium salt moiety corresponds to the general formula: (R⁵⁰)(R⁵¹)(R⁵²)(R⁵³)N⁺)E⁻ where each of R⁵⁰, R⁵¹, R⁵⁴, and R⁵⁵ are independently selected from an aliphatic group having from 1 to about 22 carbon atoms (e.g., alkyl, alkenyl); an aromatic (e.g., phenyl benzyl); alkoxy; polyoxyalkylene (e.g., polyethylene, polypropylene, and combinations thereof); acetamido; alkylamido; alkylamidoalkyl; hydroxyalkyl; aryl; araalkyl; or alkylaryl group having 1 to about 22 carbon atoms in the alkyl chain; and E⁻ is a salt-forming anion such as those selected from halogen, (e.g. chloride, bromide), acetate, citrate, lactate, glycolate, phosphate, nitrate, sulfate, and alkylsulfate (e.g., methosulfate, ethosulfate). The aliphatic groups can contain, in addition to carbon and hydrogen atoms, ether linkages, ester linkages, and other groups such as amino groups. The longer chain aliphatic groups, e.g., those of about 12 carbons, or higher, can be saturated or unsaturated. Any two of R⁵⁰, R⁵¹, R⁵⁴, and R⁵⁵ together with the nitrogen atom to which they are attached can be taken together to form a ring structure containing 5 to 6 carbon atoms, one of said carbon atoms can optionally be replaced with a heteroatom selected from nitrogen, oxygen or sulfur.

In one aspect, the quaternary ammonium moiety contains at least one nitrogen atom that is covalently linked to at least three alkyl and/or aryl substituents, and the nitrogen atom remains positively charged regardless of the environmental pH.

In one aspect, the quaternary ammonium moiety contains one nitrogen atom and at least one C₁₂ to C₂₂ alkyl group. In one aspect, the quaternary ammonium moiety contains one C₁₂ to C₂₂ alkyl group and at least two C₁ to C₅ alkyl groups (e.g., methyl, ethyl, propyl, butyl and pentyl, and combinations thereof). In one aspect, the quaternary ammonium moiety contains one C₁₂ to C₂₂ alkyl group, and three C₁ to C₅ alkyl groups (e.g., methyl, ethyl, propyl, butyl and pentyl, and combinations thereof). In one aspect, the quaternary ammonium moiety contains one C₁₂ to C₂₂ alkyl group, and two C₁ to C₅ alkyl groups (e.g., methyl, ethyl, propyl, butyl and pentyl, and combinations thereof), and one moiety containing an alkoxy; polyoxyalkylene (e.g., polyethylene, polypropylene, and combinations thereof), where the polyoxyalkylene moiety contains 3 to 100 repeating units; acetamide; alkylamido; alkylamidoalkyl; hydroxyalkyl; aryl; araalkyl; or alkylaryl group having 1 to about 22 carbon atoms in the alkyl chain, and having 6 to about 14 carbon atoms in the aryl moiety.

A number of quaternary nitrogen-containing compounds and polymers, their manufacturers and general descriptions of their chemical characteristics are found in the CTFA Dictionary and in the International Cosmetic Ingredient Dictionary, Vol. 1 and 2, 5th Ed., published by the Cosmetic Toiletry and Fragrance Association, Inc. (CTFA) (1993), the pertinent disclosures of which are incorporated herein by reference. The name assigned to the ingredients by the CTFA or by the manufacturer is used for convenience.

Non-limiting examples of monomeric quaternary ammonium compounds useful as cationic conditioners in the present technology include Acetamidopropyl Trimonium Chloride, Behenamidopropyl Ethyldimonium Ethosulfate, Behentrimonium Chloride, Behentrimonium Methosulfate, Cetethyl Morpholinium Ethosulfate, Cetrimonium Chloride, Cocoamidopropyl Ethyldimonium Ethosulfate, Dicetyldimonium Chloride, Hydroxyethyl Behenamidopropyl Dimonium Chloride, Quaternium-26, Quaternium-27, Quaternium-53, Quaternium-63, Quaternium-70, Quaternium-72, Quaternium-76 PPG-9 Diethylmonium Chloride, PPG-25 Diethylmonium Chloride, PPG-40 Stearalkonium Chloride, Isostearamidopropyl Ethyldimonium Ethosulfate, and mixtures thereof.

Cationic polymers are also useful as conditioning agents alone or in combination with the other conditioning agents described herein. Suitable cationic polymers can be synthetically derived or natural polymers can be synthetically modified to contain cationic moieties. Polymeric quaternary ammonium moiety salt containing polymers can be prepared by the polymerization of a diallylamine such as dialkyldiallylammonium salt or copolymer thereof in which the alkyl group contains 1 to about 22 carbon atoms in one aspect and methyl or ethyl in another aspect. Copolymers containing a quaternary moiety derived from a dialkyldiallylammonium salt and an anionic component derived from anionic monomers of acrylic acid and methacrylic acid are suitable conditioning agents. Also suitable are, polyampholyte terpolymers having a cationic component prepared from a derivative of diallylamine, such as a dimethyldiallylammonium salt, an anionic component derived from anionic monomers of acrylic acid or 2-acrylamido-2-methylpropane sulfonic acid and a nonionic component derived from nonionic monomers of acrylamide. The preparation of such quaternary ammonium salt moiety containing polymers can be found, for example, in U.S. Pat. Nos. 3,288,770; 3,412,019; 4,772,462 and 5,275,809, the pertinent disclosures of which are incorporated herein by reference.

In one aspect, suitable polymers include the chloride salts of the foregoing quaternized homopolymers and copolymers in which the alkyl group is methyl or ethyl, and are commercially available under the Merquat® series of trademarks from Lubrizol Advanced Materials, Inc.

A homopolymer prepared from diallyl dimethyl ammonium chloride (DADMAC) having the CTFA name, Polyquaternium-6, is available under the Merquat 100 and Merquat 106 trademark. A copolymer prepared from DADMAC and acrylamide having the CTFA name, Polyquaternium-7, is sold under the Merquat 550 trademark. Another copolymer prepared from DADMAC and acrylic acid having the CTFA name, Polyquaternium-22, is sold under the Merquat 280 trademark. The preparation of Polyquaternium-22 and its related polymers is described in U.S. Pat. No. 4,772,462, the pertinent disclosures of which are incorporated herein by reference.

Also useful is an ampholytic terpolymer prepared from a nonionic component derived from acrylamide or methyl acrylate, a cationic component derived from DADMAC or methacrylamidopropyl trimethyl ammonium chloride (MAPTAC), and an anionic component derived from acrylic acid or 2-acrylamido-2-methylpropane sulfonic acid or combinations of acrylic acid and 2-acrylamido-2-methylpropane sulfonic acid. An ampholytic terpolymer prepared from acrylic acid, DADMAC and acrylamide having the CTFA name, Polyquarternium-39, is available under the Merquat Plus 3330 trademark. Another ampholytic terpolymer prepared from acrylic acid, methacrylamidopropyl trimethyl ammonium chloride (MAPTAC) and methyl acrylate having the CTFA name, Polyquarternium-47, is available under the Merquat 2001 trademark. Still another ampholytic terpolymer prepared from acrylic acid, MAPTAC and acrylamide having the CTFA name, Polyquarternium-53, is available under the Merquat 2003PR trademark. The preparation of such terpolymers is described in U.S. Pat. No. 5,275,809, the pertinent disclosures of which are incorporated herein by reference.

Other cationic polymers and copolymers suitable as conditioners in the hair straightening compositions of the disclosed technology have the CTFA names Polyquaternium-4, Polyquaternium-11, Polyquarternium-16, Polyquaternium-28, Polyquaternium-29, Polyquaternium-32, Polyquaternium-33, Polyquaternium-35, Polyquaternium-37, Polyquaternium-44, Polyquaternium-46, Polyquaternium-47, Polyquaternium-52, Polyquaternium-53, Polyquarternium-55, Polyquaternium-59, Polyquaternium-61, Polyquaternium-64, Polyquaternium-65, Polyquaternium-67, Polyquaternium-69, Polyquaternium-70, Polyquaternium-71, Polyquaternium-72, Polyquaternium-73, Polyquaternium-74, Polyquaternium-76, Polyquaternium-77, Polyquaternium-78, Polyquaternium-79, Polyquaternium-80, Polyquaternium-81, Polyquaternium-82, Polyquaternium-84, Polyquaternium-85, Polyquaternium-87, and PEG-2-cocomonium chloride.

Exemplary cationically modified natural polymers suitable for use in the hair straightening composition include polysaccharide polymers, such as cationically modified cellulose and cationically modified starch derivatives modified with a quaternary ammonium halide moiety. Exemplary cationically modified cellulose polymers are salts of hydroxyethyl cellulose reacted with trimethyl ammonium substituted epoxide (CTFA, Polyquaternium-10). Other suitable types of cationically modified cellulose include the polymeric quaternary ammonium salts of hydroxyethyl cellulose reacted with lauryl dimethyl ammonium substituted epoxide (CTFA, Polyquaternium-24). Cationically modified potato starch having the CTFA name, Starch Hydroxypropyltrimonium Chloride, is available under the Sensomer™ CI-50 trademark, from Lubrizol Advanced Materials, Inc.

Other suitable cationically modified natural polymers include cationic polygalactomannan derivatives such as guar gum derivatives and cassia gum derivatives, e.g., CTFA: Guar Hydroxypropyltrimonium Chloride, Hydroxypropyl Guar Hydroxypropyltrimonium Chloride, and Cassia Hydroxypropyltrimonium Chloride. Guar hydroxypropyltrimonium chloride is commercially available under the Jaguar™ trade name series from Rhodia Inc. and the N-Hance trade name series from Ashland Inc. Cassia Hydroxypropyltrimonium Chloride is commercially available under the Sensomer™ CT-250 and Sensomer™ CT-400 trademarks from Lubrizol Advanced Materials, Inc.

The non-polymeric and polymeric cationic compounds can be present from about 0.05 to about 5 wt. % percent in one aspect, from about 0.1 to about 3 wt. percent in another aspect, and from about 0.5 to about 2.0 wt. % in a further aspect (based on the total weight of the composition).

### 3. Hydrocarbon Oils

The conditioning component of the hair straightening compositions of the disclosed technology can also contain hydrocarbon oil conditioners.

Suitable conditioning oils for use as conditioning agents in the compositions of the disclosed technology include, but are not limited to, hydrocarbon oils having at least about 10 carbon atoms, such as cyclic hydrocarbons, straight chain aliphatic hydrocarbons (saturated or unsaturated), and branched chain aliphatic hydrocarbons (saturated or unsaturated), including polymers and mixtures thereof. Straight chain hydrocarbon oils typically contain about 12 to 19 carbon atoms. Branched chain hydrocarbon oils, including hydrocarbon polymers, typically will contain more than 19 carbon atoms.

Specific non-limiting examples of these hydrocarbon oils include paraffin oil, mineral oil, saturated and unsaturated dodecane, saturated and unsaturated tridecane, saturated and unsaturated tetradecane, saturated and unsaturated pentadecane, saturated and unsaturated hexadecane, polybutene, polydecene, and mixtures thereof. Branched-chain isomers of these compounds, as well as of higher chain length hydrocarbons, can also be used, examples of which include highly branched, saturated or unsaturated, alkanes such as the permethyl-substituted isomers, e.g., the permethyl-substituted isomers of hexadecane and eicosane, such as 2,2,4,4,6,6,8,8-dimethyl-10-methylundecane and 2,2,4,4,6,6-dimethyl-8-methylnonane, available from Permethyl Corporation. Hydrocarbon polymers such as polybutene and polydecene. A preferred hydrocarbon polymer is polybutene, such as the copolymer of isobutylene and butene. A commercially available material of this type is L-14 polybutene from BP Chemical Company.

Liquid polyolefin conditioning oils can be used in the hair straightening compositions of the present technology. The liquid polyolefin conditioning agents are typically poly-α-olefins that have been hydrogenated. Polyolefins for use herein can be prepared by the polymerization of C₄ to about C₁₄ olefinic monomers. Non-limiting examples of olefinic monomers for use in preparing the polyolefin liquids herein include ethylene, propylene, 1-butene, 1-pentene, 1-hexene, 1-octene, 1-decene, 1-dodecene, 1-tetradecene, branched chain isomers such as 4-methyl-1-pentene, and mixtures thereof. In one aspect of the disclosed technology, hydrogenated α-olefin monomers include, but are not limited to: 1-hexene to 1-hexadecenes, 1-octene to 1-tetradecene, and mixtures thereof.

Fluorinated or perfluorinated oils are also contemplated within the scope of the present technology. Fluorinated oils include perfluoropolyethers described in European Patent 0 486 135 and the fluorohydrocarbon compounds described in WO 93/11103. The fluoridated oils may also be fluorocarbons such as fluoramines, e.g., perfluorotributylamine, fluoridated hydrocarbons, such as perfluorodecahydronaphthalene, fluoroesters, and fluoroethers.

### 4. Natural Oils

Natural oil conditioners are also useful in the practice of the disclosed technology and include but are not limited to peanut, sesame, avocado, coconut, cocoa butter, almond, safflower, corn, cotton seed, sesame seed, walnut oil, castor, olive, jojoba, palm, palm kernel, soybean, wheat germ, linseed, sunflower seed; eucalyptus, lavender, vetiver, litsea, cubeba, lemon, sandalwood, rosemary, chamomile, savory, nutmeg, cinnamon, hyssop, caraway, orange, geranium, cade, and bergamot oils, fish oils, glycerol tricaprocaprylate; and mixtures thereof. The natural oils can also be utilized as emollients.

### 5. Natural and Synthetic Waxes

Natural and synthetic wax conditioning agents can be employed in the hair straightening compositions of the disclosed technology, including but are not limited to carnauba wax, candelila wax, alfa wax, paraffin wax, ozokerite wax, olive wax, rice wax, hydrogenated jojoba wax, bees wax, modified bees wax, e.g., cerabellina wax, marine waxes, polyolefin waxes, e.g., polyethylene wax; and mixtures thereof.

### 6. Ester Oils

Ester oil conditioners include, but are not limited to, fatty esters having at least 10 carbon atoms. These fatty esters include esters derived from fatty acids or alcohols (e.g., mono-esters, polyhydric alcohol esters, and di- and tri-carboxylic acid esters). The fatty esters hereof may include or have covalently bonded thereto other compatible functionalities, such as amides and alkoxy moieties (e.g., ethoxy or ether linkages, etc.).

Exemplary fatty esters include, but are not limited to isopropyl isostearate, hexyl laurate, isohexyl laurate, isohexyl palmitate, isopropyl palmitate, decyl oleate, isodecyl oleate, hexadecyl stearate, decyl stearate, isopropyl isostearate, dihexyldecyl ad ipate, lauryl lactate, myristyl lactate, cetyl lactate, oleyl stearate, oleyl oleate, oleyl myristate, lauryl acetate, cetyl propionate, and oleyl adipate.

Other fatty esters suitable for use in the compositions of the disclosed technology are mono-carboxylic acid esters of the general formula R⁶⁰C(O)OR⁶¹, wherein R⁶⁰ and R⁶¹ are alkyl or alkenyl radicals, and the sum of carbon atoms in R⁶⁰ and R⁶¹ is at least 10 in one aspect, and at least 22 in another aspect of the disclosed technology.

Still other fatty esters suitable for use in the compositions of the disclosed technology are di- and tri-alkyl and alkenyl esters of carboxylic acids, such as esters of C₄ to C₈ dicarboxylic acids (e.g., C₁ to C₂₂ esters, preferably C₁ to C₆, of succinic acid, glutaric acid, adipic acid). Specific non-limiting examples of di- and tri-alkyl and alkenyl esters of carboxylic acids include isocetyl stearyol stearate, diisopropyl adipate, and tristearyl citrate.

Other fatty esters suitable for use in the compositions of the disclosed technology are those known as polyhydric alcohol esters. Such polyhydric alcohol esters include alkylene glycol esters, such as ethylene glycol mono and di-fatty acid esters, diethylene glycol mono- and di-fatty acid esters, polyethylene glycol mono- and di-fatty acid esters, propylene glycol mono- and di-fatty acid esters, polypropylene glycol monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters.

Specific non-limiting examples of suitable synthetic fatty esters include: P-43 (C₈ to C₁₀ triester of trimethylolpropane), MCP-684 (tetraester of 3,3 diethanol-1,5 pentadiol), MCP 121 (C₈ to C₁₀ diester of adipic acid), all of which are available from ExxonMobil Chemical Company.

The amount of hydrocarbon and natural conditioning oils, natural and synthetic waxes, and ester oil conditioning agents was can range from about 0.05 to about 10 wt. %, in one aspect, from about 0.5 to about 5 wt. % in another aspect, and from about 1 to about 3 wt. % in a further aspect (based on the total weight of the composition).

Rheology Modifiers

To provide a composition which adheres well to the hair fibers, the composition can include a rheology modifier which increases the overall viscosity of the composition. The viscosity of the composition, when applied to the hair, can be at least 400 cPs, or at least 1000 cPs, or at least 2000 cPs, or at least 3000 cPs, and can be up to 10,000 cPs.

To increase the viscosity, the composition may include one or more rheology modifiers, which can be synthetic or natural.

Examples include fatty alcohols, such as C₁₀ to C₃₂ alcohols, e.g., C₁₂ to C₂₂ alcohols, natural oils, and polymers of acrylic acid and/or methacrylic acid, such as carbomers. Exemplary natural oils include mineral oils (mainly C₁₅ to C₄₀ linear and branched aliphatic alkanes, with minor amounts of cycloalkanes), which may be sold as paraffinum liquidum.

Exemplary synthetic rheology modifiers include acrylic based polymers and copolymers. One class of acrylic based rheology modifiers are the carboxyl functional alkali-swellable and alkali-soluble thickeners (ASTs) produced by the free-radical polymerization of acrylic acid alone or in combination with other ethylenically unsaturated monomers. The polymers can be synthesized by solvent/precipitation as well as emulsion polymerization techniques. Exemplary synthetic rheology modifiers of this class include homopolymers of acrylic acid or methacrylic acid and copolymers polymerized from one or more monomers of acrylic acid, substituted acrylic acid, and salts and C₁-C₃₀ alkyl esters of acrylic acid and substituted acrylic acid. As defined herein, the substituted acrylic acid contains a substituent positioned on the alpha and/or beta carbon atom of the molecule, wherein in one aspect the substituent is independently selected from C₁₋₄ alkyl, —CN, and —COOH. Optionally, other ethylenically unsaturated monomers such as, for example, styrene, vinyl acetate, ethylene, butadiene, acrylonitrile, as well as mixtures thereof can be copolymerized into the backbone. The foregoing polymers are optionally crosslinked by a monomer that contains two or more moieties that contain ethylenic unsaturation. In one aspect, the crosslinker is selected from a polyalkenyl polyether of a polyhydric alcohol containing at least two alkenyl ether groups per molecule. Other Exemplary crosslinkers are selected from allyl ethers of sucrose and allyl ethers of pentaerythritol, and mixtures thereof. These polymers are more fully described in U.S. Pat. No. 5,087,445; U.S. Pat. No. 4,509,949; and U.S. Pat. No. 2,798,053.

In one aspect, the AST rheology modifier or thickener is a crosslinked homopolymer polymerized from acrylic acid or methacrylic acid and is generally referred to under the INCI name of Carbomer. Commercially available Carbomers include Carbopol® polymers 934, 940, 941, 956, 980, and 996 available from Lubrizol Advanced Materials, Inc. In a further aspect, the rheology modifier is selected from a crosslinked copolymer polymerized from a first monomer selected from one or more monomers of acrylic acid, substituted acrylic acid, salts of acrylic acid and salts of substituted acrylic acid and a second monomer selected from one or more C₁₀-C₃₀ alkyl acrylate esters of acrylic acid or methacrylic acid. In one aspect, the monomers can be polymerized in the presence of a steric stabilizer such as disclosed in U.S. Pat. No. 5,288,814 which is herein incorporated by reference. Some of the forgoing polymers are designated under INCI nomenclature as Acrylates/C10-30 Alkyl Acrylate Crosspolymer and are commercially available under the trade names Carbopol® 1342 and 1382, Carbopol® Ultrez 20 and 21, Carbopol® ETD 2020 and Pemulen® TR-1 and TR-2 from Lubrizol Advanced Materials, Inc.

In another aspect, the auxiliary rheology modifier can be a crosslinked, linear poly(vinyl amide/acrylic acid) copolymer as disclosed in U.S. Pat. No. 7,205,271, the disclosure of which is herein incorporated by reference.

Another class of synthetic rheology modifiers suitable for use in the composition includes hydrophobically modified ASTs, commonly referred to as hydrophobically modified alkali-swellable and alkali-soluble emulsion (HASE) polymers. Typical HASE polymers are free radical addition polymers polymerized from pH sensitive or hydrophilic monomers (e.g., acrylic acid and/or methacrylic acid), hydrophobic monomers (e.g., C₁-C₃₀ alkyl esters of acrylic acid and/or methacrylic acid, acrylonitrile, styrene), an “associative monomer”, and an optional crosslinking monomer. The associative monomer comprises an ethylenically unsaturated polymerizable end group, a non-ionic hydrophilic midsection that is terminated by a hydrophobic end group. The non-ionic hydrophilic midsection comprises a polyoxyalkylene group, e.g., polyethylene oxide, polypropylene oxide, or mixtures of polyethylene oxide/polypropylene oxide segments. The terminal hydrophobic end group is typically a C₈ to C₄₀ aliphatic moiety. Exemplary aliphatic moieties are selected from linear and branched alkyl substituents, linear and branched alkenyl substituents, carbocyclic substituents, aryl substituents, aralkyl substituents, arylalkyl substituents, and alkylaryl substituents. In one aspect, associative monomers can be prepared by the condensation (e.g., esterification or etherification) of a polyethoxylated and/or polypropoxylated aliphatic alcohol (typically containing a branched or unbranched C₈ to C₄₀ aliphatic moiety) with an ethylenically unsaturated monomer containing a carboxylic acid group (e.g., acrylic acid, methacrylic acid), an unsaturated cyclic anhydride monomer (e.g., maleic anhydride, itaconic anhydride, citraconic anhydride), a monoethylenically unsaturated monoisocyanate (e.g., α,α-dimethyl-m-isopropenyl benzyl isocyanate) or an ethylenically unsaturated monomer containing a hydroxyl group (e.g., vinyl alcohol, allyl alcohol). Polyethoxylated and/or polypropoxylated aliphatic alcohols are ethylene oxide and/or propylene oxide adducts of a monoalcohol containing the C₈ to C₄₀ aliphatic moiety. Non-limiting examples of alcohols containing a C₈ to C₄₀ aliphatic moiety are capryl alcohol, iso-octyl alcohol (2-ethyl hexanol), pelargonic alcohol (1-nonanol), decyl alcohol, lauryl alcohol, myristyl alcohol, cetyl alcohol, cetearyl alcohol (mixture of C₁₆ to C₁₈ monoalcohols), stearyl alcohol, isostearyl alcohol, elaidyl alcohol, oleyl alcohol, arachidyl alcohol, behenyl alcohol, lignoceryl alcohol, ceryl alcohol, montanyl alcohol, melissyl, lacceryl alcohol, geddyl alcohol, and C₂ to C₂₀ alkyl substituted phenols (e.g., nonyl phenol), and the like.

Exemplary HASE polymers are disclosed in U.S. Pat. Nos. 3,657,175; 4,384,096; 4,464,524; 4,801,671; and 5,292,843. In addition, an extensive review of HASE polymers is found in Gregory D. Shay, Chapter 25, “Alkali-Swellable and Alkali-Soluble Thickener Technology A Review”, Polymers in Aqueous Media—Performance Through Association, Advances in Chemistry Series 223, J. Edward Glass (ed.), ACS, pp. 457-494, Division Polymeric Materials, Washington, DC (1989), the relevant disclosures of which are incorporated herein by reference. Commercially available HASE polymers are sold under the trade names, Aculyn® 22 (INCI Name: Acrylates/Steareth-20 Methacrylate Copolymer), Aculyn® 44 (INCI Name: PEG-150/Decyl Alcohol/SMDI Copolymer), Aculyn 46® (INCI Name: PEG-150/Stearyl Alcohol/SMDI Copolymer), and Aculyn® 88 (INCI Name: Acrylates/Steareth-20 Methacrylate Crosspolymer) from Rohm & Haas, and Novethix™ L-10 (INCI Name: Acrylates/Beheneth-25 Methacrylate Copolymer) from Lubrizol Advanced Materials, Inc.

In another embodiment, acid swellable associative polymers can be used with the hydrophobically modified, cationic polymers of the disclosed technology. Such polymers generally have cationic and associative characteristics. These polymers are free radical addition polymers polymerized from a monomer mixture comprising an acid sensitive amino substituted hydrophilic monomer (e.g., dialkylamino alkyl (meth)acrylates or (meth)acrylamides), an associative monomer (defined hereinabove), a lower alkyl (meth)acrylate or other free radically polymerizable comonomers selected from hydroxyalkyl esters of (meth)acrylic acid, vinyl and/or allyl ethers of polyethylene glycol, vinyl and/or allyl ethers of polypropylene glycol, vinyl and/or allyl ethers of polyethylene glycol/polypropylene glycol, polyethylene glycol esters of (meth)acrylic acid, polypropylene glycol esters of (meth)acrylic acid, polyethylene glycol/polypropylene glycol esters of (meth)acrylic acid), and combinations thereof. These polymers can optionally be crosslinked. By acid sensitive is meant that the amino substituent becomes cationic at low pH values, typically ranging from 0.5 to 6.5. Exemplary acid swellable associative polymers are commercially available under the trade name Structure® Plus (INCI Name: Acrylates/Aminoacrylates/C₁₀-C₃₀ Alkyl PEG-20 Itaconate) from Akzo Nobel, and Carbopol® Aqua CC (INCI Name: Polyacrylates-1 Crosspolymer) from Lubrizol Advanced Materials, Inc. In one aspect, the acid swellable polymer is a copolymer of one or more C₁-C₅ alkyl esters of (meth)acrylic acid, C₁-C₄ dialkylamino C₁-C₆ alkyl methacrylate, PEG/PPG-30/5 allyl ether, PEG 20-25 C₁₀-C₃₀ alkyl ether methacrylate, hydroxy C₂-C₆ alkyl methacrylate crosslinked with ethylene glycol dimethacrylate. Other useful acid swellable associative polymers are disclosed in U.S. Pat. No. 7,378,479.

Hydrophobically modified alkoxylated methyl glucosides, such as, for example, PEG-120 Methyl Glucose Dioleate, PEG-120 Methyl Glucose Trioleate, and PEG-20 Methyl Glucose Sesquistearate, available from Lubrizol Advanced Materials, Inc., under the trade names, Glucamate® DOE-120, Glucamate™ LT, and Glucamate™ SSE-20, respectively, are also suitable as rheology modifiers.

Polysaccharides obtained from tree and shrub exudates, such as gum Arabic, gum gahatti, and gum tragacanth, as well as pectin; seaweed extracts, such as alginates and carrageenans (e.g., lambda, kappa, iota, and salts thereof); algae extracts, such as agar; microbial polysaccharides, such as xanthan, gellan, and wellan; cellulose ethers, such as ethylhexylethylcellulose, hydroxybutylmethylcellulose, hydroxyethylmethylcellulose, hydroxypropylmethylcellulose, methylcellulose, carboxymethylcellulose, hydroxyethylcellulose, and hydroxypropylcellulose; polygalactomannans, such as fenugreek gum, cassia gum, locust bean gum, tara gum, and guar gum; starches, such as corn starch, tapioca starch, rice starch, wheat starch, potato starch and sorghum starch can also be employed in the compositions herein as suitable rheology modifiers.

The rheology modifier(s) can be used alone or in combination and may be present in the composition, on an actives basis, at a total concentration of 0.001-50 wt. %, e.g., at least 0.1 wt. %, or at least 1 wt. %, such as up to 20 wt. %, or up to 10 wt. %, or up to 3 wt. %, based on the total weight of the composition.

### pH Modifiers

The pH of the composition can range from to 1.5 to 9.5 in one aspect, at least 4.5 in a second aspect, at least 5.5 a third aspect, at least 6.5 in a fourth aspect, at least 7.0 in a fifth aspect, at least 7.5 in a sixth aspect, at least 8.0 in a seventh aspect, at least 8.5 in an eighth aspect, at least 9.0 in a ninth aspect, and at least 9.5 in a tenth aspect. To provide the desired pH, the composition may be adjusted with one or more pH modifiers selected from organic and inorganic acids and bases.

The pH of the composition can be adjusted with any combination of acidic and/or basic pH adjusting agents known to the art. Acidic materials include organic acids and inorganic acids, in particular, monocarboxylic acids, dicarboxylic acids, and tricarboxylic acids, for example, acetic acid, citric acid, tartaric acid, alpha-hydroxy acids, beta-hydroxy acids, salicylic acid, lactic acid, malic acid, glycolic acid, amino acids, and natural fruit acids, or inorganic acids, for example, hydrochloric acid, nitric acid, sulfuric acid, sulfamic acid, phosphoric acid, and combinations thereof.

Basic materials include inorganic and organic bases, and combinations thereof. Examples of inorganic bases include but are not limited to the alkali metal hydroxides (e.g., potassium hydroxide, sodium hydroxide) and alkali metal carbonates (e.g., potassium carbonate, sodium carbonate), and alkali metal salts such as sodium borate (borax), sodium phosphate, sodium pyrophosphate, and the like; and mixtures thereof. Examples of organic bases include ammonium hydroxide, triethanolamine (TEA), diisopropanolamine, triisopropanolamine, aminomethyl propanol, dodecylamine, cocamine, oleamine, morpholine, triamylamine, triethylamine, tetrakis(hydroxypropyl)ethylenediamine, L-arginine, aminomethyl propanol, tromethamine (2-amino 2-hydroxymethyl-1,3-propanediol), and PEG-15 cocamine

### Preservatives

In one aspect, any preservative suitable for use in personal care can be used in the composition for straightening hair. Suitable preservatives include polymethoxy bicyclic oxazolidine, methyl paraben, propyl paraben, ethyl paraben, butyl paraben, benzyltriazole, DMDM hydantoin (also known as 1,3-dimethyl-5,5-dimethyl hydantoin), imidazolidinyl urea, phenoxyethanol, phenoxyethylparaben, methylisothiazolinone, methylchloroisothiazolinone, benzoisothiazolinone, triclosan, and suitable polyquaternium compounds as disclosed above (e.g., Polyquaternium-1).

In another aspect, acid based preservatives are useful in the exemplary compositions. The use of acid based preservatives facilitates the formulation of products in the low pH range. Lowering the pH of a formulation inherently provides an inhospitable environment for microbial growth in addition to being suited to the straightening process. Moreover, formulating at low pH enhances the efficacy of acid based preservatives, and affords a personal care product which maintains an acidic pH balance on the skin. Any acid based preservative that is useful in personal care products can be used in the exemplary compositions. In one aspect the acid preservative is a carboxylic acid compound represented by the formula: R³C(O)OH, wherein R³ represents hydrogen, a saturated and unsaturated hydrocarbyl group containing 1 to 8 carbon atoms or C₆ to C₁₀ aryl. In another aspect, R³ is selected from a hydrogen, a C₁ to C₈ alkyl group, a C₂ to C₈ alkenyl group, or phenyl. Exemplary acids are, but are not limited to, formic acid, acetic acid, propionic acid, sorbic acid, caprylic acid, and benzoic acid, and mixtures thereof.

In another aspect, suitable acids include but are not limited to, oxalic acid, succinic acid, glutaric acid, adipic acid, azelaic acid, maleic acid, fumaric acid, lactic acid, glyceric acid, tartronic acid malic acid, tartaric acid, gluconic acid, citric acid, ascorbic acid, salicylic acid, phthalic acid, mandelic acid, benzilic acid, and mixtures thereof.

Salts of the foregoing acids are also useful as long as they retain efficacy at low pH values. Suitable salts include the alkali metal (e.g., sodium, potassium, calcium) and ammonium salts of the acids enumerated above.

The acid based preservatives and/or their salts can be used alone or in combination with non-acidic preservatives typically employed in personal care, home care, health care, and institutional and industrial care products.

The preservatives may comprise from 0.01 wt. % to 3.0 wt. % in one aspect, or from 0.1 wt. % to 1 wt. %, or from 0.3 wt. % to 1 wt. %, of the total weight of the hair straightening composition.

### Chelating Agents

Chelating agents can be employed to stabilize the composition against the deleterious effects of metal ions. When utilized, suitable chelating agents include EDTA (ethylene diamine tetraacetic acid) and salts thereof such as disodium EDTA, citric acid and salts thereof, cyclodextrins, and the like, and mixtures thereof.

Such suitable chelating agents can comprise 0.001 wt. % to 3 wt. %, such as 0.01 wt. % to 2 wt. %, or 0.01 wt. % to 1 wt. % of the total weight of the hair straightening composition.

### Propellants

Where desired, any known aerosol propellant can be utilized to deliver the hair straightening compositions onto the surface of the hair to be straightened, e.g., for dispensing the hair straightening and/or hair straightening adjuvant component in the form of an aerosol spray, mousse or gel. Exemplary propellants include lower boiling hydrocarbons such as C₃-C₆ straight and branched chain hydrocarbons. Exemplary hydrocarbon propellants include propane, butane, isobutene, and mixtures thereof. Other suitable propellants include ethers, such as, dimethyl ether, hydrofluorocarbons, such as, 1,1-difluoroethane, and compressed gases, such as air and carbon dioxide.

In one aspect, these compositions can contain from 0.1 wt. % to 60 wt. %, or 0.5 to 35 wt. % propellant, based on the total weight of the composition.

### Botanicals

The hair straightening composition can contain one or more botanical agents. Suitable botanical agents can include, for example, extracts from Echinacea (e.g., sp. angustifolia, purpurea, pallida), yucca glauca, willow herb, basil leaves, Turkish oregano, carrot root, grapefruit, fennel seed, rosemary, tumeric, thyme, blueberry, bell pepper, blackberry, spirulina, black currant fruit, tea leaves, such as for, example, Chinese tea, black tea (e.g., var. Flowery Orange Pekoe, Golden Flowery Orange Pekoe, Fine Tippy Golden Flowery Orange Pekoe), green tea (e.g., var. Japanese, Green Darjeeling), oolong tea, coffee seed, dandelion root, date palm fruit, gingko leaf, green tea, hawthorn berry, licorice, apricot kernel, sage, strawberry, sweet pea, tomato, sunflower seed extract, sandalwood extract, grape seed, aloe leaf, vanilla fruit, comfrey, arnica, Centella asiatica, cornflower, horse chestnut, ivy, Macadamia ternifolia seed, magnolia, oat, pansy, skullcap, seabuckthorn, white nettle, and witch hazel. Botanical extracts may also include, for example, chlorogenic acid, glutathione, glycrrhizin, neohesperidin, quercetin, rutin, morin, myricetin, absinthe, and chamomile.

In one aspect, the hair straightening composition can contain from about 0.01 to about 10 wt. % of one or more of the botanical extracts set forth above, from about 0.05 to about to about 5 wt. % in another aspect, from about 0.1 to about 3 wt. % in still another aspect, and from about 0.5 to about 1 wt. % in a further aspect (based on the total weight of the composition.

### Amino Acids

The hair straightening composition provided herein can contain one or more non-guanidine moiety containing amino acids. Examples of amino acids that can be used include, without limitation, capryl keratin amino acids, capryl silk amino acids, jojoba amino acids, keratin amino acids, palmitoyl keratin amino acids, palmitoyl silk amino acids, sodium cocoyl amino acids, sodium cocoyl silk amino acids, and sweet almond amino acids.

The hair straightening composition can include an appropriate amount of amino acid(s). The amount of amino acid ranges from about 0.001 to about 5 wt. % in one aspect, from about 0.01 percent to about 3 wt. % in another aspect, from about 0.1 to about 2 wt. % in still another aspect, and from about 0.5 to about 1 wt. % in a further aspect, based on the total weight of the composition.

### Vitamins

The hair straighening composition can contain one or more vitamins. Examples of vitamins that can be used include, without limitation, niacinamide, sodium starch octenylsuccinate, calcium pantothenate, maltodextrin, sodium ascorbyl phosphate, tocopheryl acetate, pyridoxine HCl, silica, panthenol (e.g., Pro Vitamin B5), phytantriol, calcium pantothenate (e.g., vitamin B5), vitamin E, and vitamin E esters (e.g., tocopheryl acetate, tocopheryl nocotinate, tocopheryl palmitate, or tocopheryl retinoate).

A hair care composition provided herein can include any amount of vitamin(s). The amount of vitamin(s) can range from about 0.05 to about 10 wt. % in one aspect, from about 0.1 to about 5 wt. % in another aspect, from about 0.5 to about 3 wt. % in still another aspect, and from about 0.75 to about 1 wt. % in a further aspect, based on the total weight of the composition.

### Fragrances and Perfumes

Fragrance and perfume components that may be used in the exemplary composition to mask the odor of any of the various components in the hair straightening composition or to give the composition an aesthetically pleasing fragrance. In one aspect, suitable fragrances and perfumes include natural and synthetic fragrances, perfumes, scents, and essences and any other substances which emit a fragrance. As the natural fragrances, there are those of vegetable origin, such as oil extracts from flowers (e.g., lily, lavender, rose, jasmine, neroli, ylang-ylang), stems and leaves (geranium, patchouli, petitgrain, peppermint), fruits (aniseed, coriander, fennel, mace, needle juniper), fruit skin (bergamot, lemon, orange), roots (angelica, celery, cardamom, costus, iris, sweet flag), woods (pine tree, sandalwood, guaiacum wood, cedar, rosewood, cinnamon), herbs and grasses (tarragon, lemongrass, sage, thyme), needles and twigs (spruce, pine, European red pine, stone pine), and resins and balsam (galbanum, elemi, benzoin, myrrh, frankincense, opopanax), and those of animal origin, such as musk, civet, castoreum, ambergris, or the like, and mixtures thereof.

Examples of synthetic fragrances and perfumes are the aromatic esters, ethers, aldehydes, ketones, alcohols, and hydrocarbons including benzyl acetate, phenoxyethyl isobutylate, p-tert-butylcyclohexyl acetate, linalyl acetate, dimethylbenzylcarbinyl acetate, phenylethyl acetate, linalyl benzoate, benzyl formate, ethylmethylphenyl glycinate, allylcyclohexyl propionate, styralyl propionate, and benzyl salicylate; benzylethyl ether; straight chain alkanals having 8 to 18 carbon atoms, citral, citronellal, citronellyloxyaldehyde, cyclamen aldehyde, hydroxycitronellal, lilial, and bougeonal; ionone compounds, α-isomethyl ionone, and methyl cedryl ketone; anethole, citronellol, eugenol, isoeugenol, geraniol, lavandulol, nerolidol, linalool, phenylethyl alcohol, and terpineol, alpha-pinene, terpenes (e.g., limonene), and balsams, and mixtures thereof.

The amount of fragrance agent or perfume employed can be any amount suitable to mask a particular odor or to impart a desired aesthetically pleasing aroma, fragrance or scent. In one aspect, the amount of fragrance agent can range from about 0.05 to about 10 wt. %, from about 0.1 to about 5 wt. % in another aspect, from about 0.5 to about 3.5 wt. % in still another aspect, and from about 1 to about 2.5 wt. % in a further aspect, based on the total weight of the composition.

### Hair Fixing Agents

Hair fixing agents may be included in addition to the exemplary thermally-activated agent, including polymer fixatives such as 3-aminopropyl methyl, dimethyl, reaction products of silicones and siloxanes with 2-ethyl-4,5-dihydrooxazole homopolymer, ethyl sulfates, such as Polysilicone-9, and film-forming polymers such as polyacrylic acid and sodium polyacrylate polymer fixatives, such as Fixate™ RSP available from Lubrizol Corp.

The hair fixing agent may be present in the composition at from 0.001 wt. % to 20 wt. %, such as at least 0.1 wt. %, or up to 5 wt. %.

### Buffer Agents

Buffering agents can be used in the exemplary compositions. Suitable buffering agents include alkali or alkali earth metal carbonates, phosphates, bicarbonates, citrates, borates, acetates, acid anhydrides, succinates, and the like, such as sodium phosphate, sodium citrate, sodium acetate, sodium bicarbonate, and sodium carbonate.

Other auxiliary components useful herein may include UV-absorbers, such as benzophenone-4.

Additionally, the selected process, such as contact time, heating temperature, and the speed at which the heating device is passed through the hair (glide rate) may also influence the selection of the composition components and their amounts, as demonstrated by the examples below.

The semi-permanent hair straightening composition can be provided and dispensed from suitable package forms, such as pressurized and non-pressurized containers, such as cans, bottles, packets, ampoules, jars, tubes, and the like. Spray compositions can be dispensed from finger-actuated pump devices, either as pressurized aerosol sprays, mousses, spritzes, and foams containing propellant, or as non-pressurized, mechanically propelled sprays and foams.

The exemplary composition can be utilized on hair to impart an excellent straightening effect by using heat. The treatment method includes first coating the hair with the composition. This may be performed with any suitable applicator, such as a brush, comb, sponge, pad, cloth, fingers e.g., while wearing gloves, to coat the hair fibers with the composition. The composition is left in contact with the hair for a sufficient contact time period for the thermally-activated agent to penetrate into the fibers, such as several minutes, e.g., at least five or at least ten minutes, or at least twenty minutes, or at least thirty minutes, or at least forty minutes, or at least 45 minutes, and up to sixty Minutes. The optimum contact time may vary, depending on factors such as the concentration of thermally-activated agent, the selected guanidine moiety containing adjuvant and the temperature of the heating device used to contact the hair in the heating stage.

The heat may be applied with a heating device which provides a temperature sufficient to activate the thermally-activated agent of Structure (1), such as a temperature of at least 185° C., or at least 190° C., or at least 200° C., or at least 205° C. or at least or about 210° C., or at least 215° C., or up to 220° C., or up to 225° C. or up to 230° C. The heating device is retained in contact with the hair for sufficient time to effect the desired configuration modification, such as an increase in length of a hair tress of at least 5%, or at least 10%. The time depends on a number of factors such as the moisture content of the hair, temperature of the heating device, concentration of the thermally-activated hair straightening composition and so forth, but is generally at least 10 seconds, or at least 1 or 2 minutes, in total, for each centimeter length of hair. Example heating devices include flat or round irons, microwave generators, sources of infrared radiation, and the like. In the case of flat or round iron, for example, the heating device has at least one surface (which is brought into contact with the fibers) which is raised, e.g., with an electric power source, to a surface temperature of at least 180° C., or at least 190° C., or at least 200° C., or at least 205° C. or at least or about 210° C. and up to 230° C. or up to 225° C., or up to 220° C.

The temperature of the hair tress can be measured, for example, with a thermocouple positioned within the hair tress and may reach a temperature of at least 150° C. or at least 170° C. during the heating with the heating device.

Optionally, the hair can be dried to some extent after treatment with the straightening composition and prior to application of heat to raise the temperature of the keratin fibers and/or to avoid substantial release of steam during the heating stage. For example, partial drying may be achieved by blow drying with a hairdryer, a drying hood at a temperature of about 100° C., by free drying, wiping with a towel, etc. For example, the moisture content of the composition may be reduced to 10 wt. % or less, such as about 2 wt. %, prior to applying heat with the heating device. In general, the drying is performed to remove solvent while retaining at least a portion, or all, of the applied thermally-activated agent on the hair. For this reason also, the hair is not rinsed (e.g., with water or other aqueous solution) between the applying and the heating stages in the exemplary embodiment.

The end of the iron that comes into contact with the hair generally has two flat surfaces. These two flat surfaces may be metallic or ceramic. They may be smooth or notched. The application of the iron may be performed by successive passes down the length of the hair separated by a few seconds, or by gradual moving or sliding along locks, etc. The application of the iron in the exemplary process is performed by continuous movement from the root to the end, in one or more passes, such as at least five passes, or at least 10 passes, or at least 12 passes, or at least 15 passes. The speed at which the heating device such as a flat iron is passed from the root to the tip of the hair can be at least 1 cm/sec., or at least 2 cm/sec./pass, or at least about 2.5 cm/sec./pass, or at least 3 cm/sec./pass, or at least 4 cm/sec./pass.

Without wishing to be bound by any particular theory, it is suggested that the hot flat iron softens hard α-keratins and straightens the hair fiber and synergistically, the heat imparted by the flat iron activates a reaction (e.g., a cross-linking or other modification reaction) between the cyclic carbonates (or other thermally-activated agent according to Structure (I)) and the amine groups of the hair fibers, possibly resulting in N,N′-disubstituted urea linkages. This results in a fixation of the newly formed shape.

Results of tests suggest that ethylene carbonate, among others, such as in an aqueous solution, can readily penetrate the hair fibers and react with hard proteinaceous materials via a flat iron at a temperature higher than 185° C. or such as about 210° C. The curly hair-tresses used in the tests, once straightened, can withstand up to 12 shampoo-wash cycles in one aspect, up to 24 shampoo-wash cycles in another aspect, up to 30 shampoo-wash cycles in still another aspect, up to 36 shampoo-wash cycles in a further aspect, and up to 50 shampoo-wash-cycles in a still further aspect. The straightened hair-tresses look shiny and feel smooth and silky without malodor. In one embodiment, propylene carbonate and glycerol carbonate can be used in combination with other thermally-activated agents of Structure (I) or other keratin cross-linking agents. In addition, propylene carbonate and glycerol carbonate are excellent solvents/diluents for solubilizing ethylene carbonate.

Alkylene carbonates, in particular, ethylene carbonate, in combination with an adjuvant containing a guanidine moiety have been discovered to be an effective hair straightening composition. In brief, applying an composition containing ethylene carbonate and a guanidine containing moiety to curly or wavy hair followed by a hot flat iron treatment (e.g., at 210° C.) can effectively modify (straighten) its shape as desired and that this shape can last for days to weeks (e.g., 50 shampoos or equivalent to about 4 months) without returning to its original curly or wavy state. This compares very favorably with formaldehyde based formulations.

By way of example, the composition and process provides some or all of the following attributes:
1. Activated via a flat iron heating device to create straight, smooth, sleek hair.
2. Retains style for at least 24 shampoo-washes and up to 8 or 12 weeks (for a consumer using 3 shampoo-washes/week), and can be up to 50 shampoos or equivalent to about 4 months, or longer.
3. Resistant to high humidity.
4. Less heat damage to the hair than existing, formaldehyde-based hair straightening compositions.
5. Low or no volatile organic compounds (VOCs).
6. Formaldehyde-free.
7. Applicable to all hair-types, including natural and chemically-treated hair.
8. Suitable for home use as well as salon application.

Without intending to limit the scope of the exemplary embodiment, the following examples demonstrate the effectiveness of example compositions.

### EXAMPLES

In the following examples, the hair type and curliness is based on the definition published by L'Oreal (Roland de la Mettrie, et al. “Shape Variability and Classification of Human Hair: A Worldwide Approach,” Human Biology, Jun. 2007). This assigns a number to the hair from I-VIII, with I being the straightest and VIII representing dense, very curly hair. In general, the hair samples used herein were initially classed as type III-IV (curly). After thermal straightening, the hair-tress typically becomes type I (straight). However, if after a 3-shampoo-wash-cycle, the hair-tress relaxes to type II, this can be considered as wavy (relaxed).

To simulate human hair on the head, curly Brazilian dark brown virgin hair tresses (classified as type III-IV) were obtained from International Hair Importers and Products Inc. Each individual hair-tress is about 2.5 g in weight and is clamped at the root end. Each tress has approximate dimensions of 17 cm in length (in the natural curly state) and 2 cm in width at the clamped end.

The thermally-activated hair straightening compositions are applied to the hair tresses by solution dipping or cream chassis application, followed by heat-treating and then evaluated for wash fastness.

### Examples 1-17

### Solution Dipping Application

### Hair Tress Preparation

Prior to dipping, each hair tress is shampooed with a commercial cleansing shampoo (e.g., VO5™ or Suave™ shampoo). The shampoo (2.5 g) is applied to each hair tress and gently massaged into the tress for 30 seconds followed by a thorough rinsing for 60 seconds under running warm tap water (35-38° C.). Each washed hair tress is allowed to dry overnight at room temperature (23° C.) in 50% relative humidity conditions. The dried hair tresses are then wetted under warm running tap water (35-38° C.) for 30 seconds. Excess water is removed by pinching each tress between the gloved index and middle fingers and gently pulling the tress through the gap of the fingers from the root end to the tip.

### Solution Dipping Treatment

A solution of the test hair straightening compositions is prepared in an aqueous solvent system by dissolving the desired test quantity of alkylene carbonate (straightener) and guanidine moiety containing compound (adjuvant) indicated in Table 1 in deionized water. If desired, the pH of the solution was adjusted with a suitable acid or base (e.g., citric acid or sodium carbonate). The dipping solution was allowed to equilibrate to ambient room temperature (23° C.). The damp hair tress was immersed completely in the dipping solution for the desired contact time (Table 1). Then, the treated hair tress was removed from the solution, the excess amount of material squeezed out by pinching each tress between the index finger and the middle finger and gently pulling the tress through the gap of the gloved fingers from the root end to the tip. The hair tress was sandwiched between two paper towels while applying slight pressure to remove any remaining solution on the surface of the tress. Each tress was blow-dried to at least 95% dryness before thermal-straightening.

Dipping solution compositional information, test parameters and evaluation results are presented in Table 1.

TABLE 1

| | | | Straightening | | | | | | | |
|---|---|---|---|---|---|---|---|---|---|---|
| Ex. | EC | Straightening | Adjuvant (wt. | | D.I. Water | Contact Time | Shampoo | Flat Iron | Type | |
| No. | (wt. %) | Adjuvant | %) | pH | (wt. %) | (min.) | Cycles | Temp. (° C.) | Rating² | Evaluation |
| 1 | 25 | Guanidine | 5 | 6.2 | q.s. to 100 | 20 | 24 | ~210 | I | S |
| | | Hydrochloride | | | | | | | | |
| 2 | 25 | Guanidine | 5 | 8.7 | q.s. to 100 | 20 | 24 | ~210 | I | S |
| | | Carbonate | | | | | | | | |
| 3 | 27 | Guanidine | 3 | 8.3 | q.s. to 100 | 20 | 24 | ~210 | I | S |
| | | Carbonate | | | | | | | | |
| 4 | 23 | Guanidinopropionic | 7 | 6.8 | q.s. to 100 | 20 | 24 | ~210 | I | S |
| | | Acid | | | | | | | | |
| 5 | 25 | Guanidinopropionic | 5 | 6.9 | q.s. to 100 | 20 | 24 | ~210 | I | S |
| | | Acid | | | | | | | | |
| 6 | 20 | Creatinine | 10 | 8.5 | q.s. to 100 | 20 | 24 | ~210 | I | S |
| 7 | 25 | Creatinine | 5 | 8.4 | q.s. to 100 | 20 | 24 | ~210 | I | S |
| 8 | 25 | Creatine Ethyl | 5 | 7.9 | q.s. to 100 | 20 | 24 | ~210 | I | S |
| | | Ester Hydrochloride | | | | | | | | |
| 9 | 25 | 1,5,7- | 5 | 8.5 | q.s. to 100 | 20 | 24 | ~210 | I | S |
| | | Triazabicyclo[4.4.0] | | | | | | | | |
| | | dec-5-ene | | | | | | | | |
| 10 | 25 | 1,5,7- | 3 | 8.0 | q.s. to 100 | 20 | 24 | ~210 | I | S |
| | | Triazabicyclo[4.4.0] | | | | | | | | |
| | | dec-5-ene | | | | | | | | |
| 11 | 0¹ | Guanidine | 45 | 5.4 | q.s. to 100 | 20 | 24 | ~210 | III-IV | C |
| | | Hydrochloride | | | | | | | | |
| 12 | 0¹ | Guanidine | 45 | 9.8 | q.s. to 100 | 20 | 24 | ~210 | III-IV | C |
| | | Carbonate | | | | | | | | |
| 13 | 25¹ | L-Ornithine | 5 | 6.3 | q.s. to 100 | 20 | 24 | ~210 | III-IV | C |
| 14 | 25¹ | L-Citrulline | 5 | 7.2 | q.s. to 100 | 20 | 24 | ~210 | III-IV | C |
| 15 | 30¹ | None | 0 | 6.7 | q.s. to 100 | 30 | 3 | ~210 | III | C |
| 16 | 30¹ | None | 0 | 6.7 | q.s. to 100 | 45 | >24 | ~210 | I-II | S |
| 17 | 30¹ | None | 0 | 6.7 | q.s. to 100 | 30 | 12 | ~226 | I-II | S |

¹Comparative Example
²Classified according to Shape Variability and Classification of Human Hair: A Worldwide Approach, Human Biology, vol. 79, No. 3 June 2007
EC = Ethylene Carbonate
S = Straight
C = Curley

### Thermal Straightening

For thermal straightening a commercial flat iron heating device, sold under the tradename Nano Titanium™ by BaByliss PRO, was used. The protocol was as follows:
1. The flat iron is set at a target temperature of approximately 210° C.
2. The heated flat iron is drawn from the top root end (clamped end) to the bottom of the tress in approximately 10-12 seconds (approx. 2 2.3 cm/sec. glide rate).
3. The thermal process is repeated for the desired straightening effect (e.g., 10-12 passes at 208-210° C.).
4. After the thermal straightening treatment, the hair-tress is allowed to cool to room temperature and evaluated for wash-fastness.

### Wash Fastness

Shampoo wash fastness (the ability to maintain a straight hold configuration following numerous shampoo wash-rinse cycles) is carried out by shampooing and rinsing the treated hair tress as described above for the hair tress preparation step. Twenty-four consecutive shampoo wash-rinse cycles were carried out. After every 3^{rd} shampoo wash-rinse cycle (up to 12) a drying step was employed (as described for the hair tress preparation step). After the twelfth shampoo wash-rinse cycle, the drying step was employed after every 6th shampoo wash-rinse cycle. After the 24th shampoo wash-rinse cycle each hair tress was dried according to the protocol above and evaluated.

The results demonstrate that straightening can be achieved by selection of contact time, flat iron temperature, and concentration of thermally the activated agents. Hair tresses treated with the comparative compositions comprising ethylene carbonate and the amino group rich compounds (non-guanidine moiety containing compounds), L-ornithine and L-citrulline, under the same contact time and heat settings failed to maintain a straight configuration after 24 shampoo wash-rinse cycles. Hair tresses treated with the control formulations (30 wt. % ethylene carbonate with no guanidine moiety containing adjuvant compounds) needed substantially increased contact times and/or increased flat iron temperatures in order to maintain a straight configuration following repeated shampoo wash-rinse cycles.

Ethylene carbonate exhibits enhanced efficacy (e.g., shorter contact times and reduced flat iron temperatures) when utilized in combination with a guanidine moiety containing straightening adjuvant.

### Examples 18-36

### Cream Chassis Application

Cream based hair straightening formulations were prepared by separately formulating the guanidine moiety containing adjuvant in a cream base comprising the ingredients and amounts indicated in Table 2 (Part 1), and a solution of an alkylene carbonate hair straightening component comprising the ingredients and amounts indicated in Table 3. The cream base was formulated to contain 7.5 wt. % or 11.0 wt. % of L-arginine, and the alkylene carbonate solution was prepared by dissolving ethylene carbonate (EC) in propylene carbonate (PC) (75:25 EC:PC wt./wt.) (Part 2).

Immediately before application to the hair tress, a final cream based hair straightening composition was prepared by combining a requisite amount of the cream based guanidine moiety containing adjuvant component (Part 1) with a requisite amount of the ethylene carbonate hair straightener solution (Part 2) to obtain the wt. % of active components indicated in Table 6, and mixing the combined components with a spatula until a homogeneous cream was obtained.

TABLE 2
(Part 1 Adjuvant Cream)

| | | |
|---|---|---|
| Component | INCI Name | Wt. % |

Phase A

| | | |
|---|---|---|
| 1 | Ceteareth-20 (emulsifier) | 0.3 |
| 2 | Petrolatum (emollient) | 1.5 |
| 3 | Methyl Glucose Sesquistearate | 1.7 |
| | (Glucate ™ SS Emulsifier, | |
| | Lubrizol Advanced Materials, | |
| | Inc.) | |
| 4 | Glyceryl Stearate (emulsifier) | 2.0 |
| 5 | Parafinium Liquidium | 2.0 |
| | (emollient) | |
| 6 | Cetearyl Alcohol¹ | 4.5 |
| | (emulsifier/conditioning agent) | |

Phase B

| | | |
|---|---|---|
| 7 | Aqua (deionized water diluent) | q.s. to 100 |
| 8 | Methyl Gluceth-20 (Glucam ™ | 1.5 |
| | E-20 Humectant, Lubrizol | |
| | Advanced Materials, Inc.) | |

Phase C

| | | |
|---|---|---|
| 9 | L-Arginine | 7.5 or 11.0 |
| 10 | Aqua (deionized water diluent) | 30.0 |

Phase E

| | | |
|---|---|---|
| 11 | Methylchloroisothiazoline (and) | 0.05 |
| | Methylisothiazoline (Euxyl ™ | |
| | K120 Preservative, Schülke | |
| | Inc.) | |
| 12 | Cyclopenasiloxane (Dow | 2.0 |
| | Corning ™ DC 245 | |
| | decamethylcyclopentasiloxane | |
| | conditioning agent) | |
| 13 | Polyquaternium-47 | 0.5 |
| | (Merquat ™ 2001 Conditioning | |
| | agent, Lubrizol Advanced | |
| | Materials, Inc.) | |
| 14 | Aqua (deionized water diluent) | 2.0 |

Phase F

| | | |
|---|---|---|
| 15 | Citric Acid (50% aqueous | q.s. to pH 8.5-9.0 |
| | wt./wt.) | |

¹30/70 (wt./wt.) blend of Cetyl and Stearyl Alcohols

The components of Phase A and Phase B were combined in separate containers. Each phase was heated to 70-75° C. under gentle agitation until homogeneous. Phase A was then added to Phase B under continuous agitation for approximately 5 min. to obtain a homogeneous emulsion. The emulsion was allowed to cool to 50° C. while stirring. Phase C was prepared by dissolving the hair straightening adjuvant compound in deionized water. Optionally, the water can be heated to 40-45° C. to aid in the dissolution of the adjuvant in the water phase. Once the adjuvant compound was fully dissolved, Phase C was added to the Phase AB emulsion and mixed until homogeneous. The Phase D components were then added to the Phase ABC emulsion and mixed until homogeneous. Phase E was prepared by dissolving the Polyquaternium component in deionized water followed by adding the solution to the Phase ABCD emulsion and mixing well until homogeneous.

The final pH of the adjuvant cream emulsion was adjusted by adding Phase F (citric acid) to the Phase ABODE emulsion to obtain a pH ranging from 8.5-9.0. The Brookfield viscosity was measured by employing a Brookfield rotating spindle viscometer, Model RVT (Brookfield Engineering Laboratories, Inc.), at about 20 revolutions per minute (rpm), using a no. 5 spindle for 1 minute at ambient room temperature of about 25° C., and was evaluated to be at about 7,200 mPa·s.

The hair straightener component was prepared by dissolving ethylene carbonate in propylene carbonate diluent and heating to 40° C. under gentle stirring until the ethylene carbonate was dissolved. The amounts of components are set forth in Table 3.

TABLE 3
(Part 2 Hair Straightener)

| Component | Wt. % |
|---|---|
| Ethylene Carbonate | 75 |
| Propylene Carbonate | 25 |

### Hair Tress Preparation

Prior to applying the hair straightening cream to the hair tress, each tress was shampooed with an alkaline pH cleansing shampoo. The alkaline cleansing shampoo was formulated from the components set forth in Table 4.

TABLE 4

| | | | |
|---|---|---|---|
| Component | INCI Name | Trade Name | Wt. % |

Phase A

| | | | |
|---|---|---|---|
| 1 | Aqua (deionized water) | Diluent | q.s. to 100 |
| 2 | Cocamide MEA | Amidex ™ CME Nonionic | 2.0 |
| | | Surfactant (Lubrizol | |
| | | Advanced Materials, Inc.) | |

Phase B

| | | | |
|---|---|---|---|
| 3 | PEG-120 Methyl Glucose | Glucamate ™ LT Thickener | 1.0 |
| | Trioleate (and) Propylene | (37-43% Actives), Lubrizol | |
| | Glycol (and) Water | Advanced Materials, Inc. | |
| 4 | Cocamidopropyl Betaine | Chembetaine ™ CAD | 2.0 |
| | | Amphoteric Surfactant (34- | |
| | | 36% Actives), Lubrizol | |
| | | Advanced Materials, Inc. | |
| 5 | Sodium Laureth Sulfate | Sulfochem ™ ES-2PK | 25.0 |
| | | Anionic Surfactant (27.5% | |
| | | Actives), Lubrizol Advanced | |
| | | Materials, Inc. | |

Phase C

| | | | |
|---|---|---|---|
| 6 | Methylchloroisothiazoline | Euxyl ™ K120 Preservative, | 0.05 |
| | (and) Methylisothiazoline | Schülke Inc. | |

Phase D

| | | | |
|---|---|---|---|
| 7 | Sodium Hydroxide 18% | pH Adjuster | q.s. to desired pH |
| | aqueous (wt./wt.) | | |

Phase E

| | | | |
|---|---|---|---|
| 8 | Potassium Chloride | Viscosity Adjuster | 0.65 |

Phase A was prepared by dissolving the Cocamide MEA surfactant in heated deionized water (75° C.) with stirring until homogeneousPhase B components are added to Phase A in the listed order with stirring until each component was dissolved and homogeneously mixed. After mixing Phase AB is allowed to cool to ambient room temperature (23° C.) while mixing is maintained. Phase C is added to Phase AB and mixed well. The pH of the composition was adjusted by the adding Phase D (sodium hydroxide) to obtain a final pH ranging from 9-9.5. Phase E is then added to Phase ABCD and mixed well. The Brookfield viscosity of the alkaline shampoo was measured by employing a Brookfield rotating spindle viscometer, Model RVT (Brookfield Engineering Laboratories, Inc.), at about 20 revolutions per minute (rpm), using a no. 5 spindle for 1 minute at ambient room temperature of about 23° C., and was found to be 12,000 mPa·s.

The alkaline shampoo (2.5 g) was applied to each hair tress and gently massaged into the tress for 30 seconds followed by a thorough rinsing for 60 seconds under running warm tap water (35-38° C.). Each washed hair tress was allowed to dry overnight at room temperature (23° C.) in 50% relative humidity conditions. The dried hair tresses were then wetted under running tap water (35-38° C.) for 30 seconds. Excess water was removed by pinching each tress between the gloved index and middle fingers and gently pulling the tress through the gap of the fingers from the root end to the tip. Each tress was sandwiched between two paper towels and gently pressed with a hand to absorb excess water.

### Cream Treatment

To each of the hair tresses prepared above 2 g of the hair straightening cream formulation prepared by combining Part 1 with Part 2 was evenly applied and thoroughly massaged throughout each tress. The cream was allowed to contact the hair for 20 min. before the tress was combed with a fine toothed comb to remove excess product. The tress was then blow dried and combed to smooth the tress before the flat iron heat treatment to thermally straighten the hair.

Using the flat iron protocol employed for the solution dipping test, each tress was subjected to 10 passes of the flat iron at a glide speed of 2 to 2.3 cm/sec. The temperature of the flat iron was set to 210° C.

Following thermal straightening with the flat iron, 2 to 3 g of a neutralizing/conditioning cream masque was applied to each hair tress. The neutralizing masque was evenly massaged into each hair tress followed by thoroughly rinsing the tress with warm water (30-35° C.) until the residual masque was rinsed away. The cream masque was formulated from the components and amounts set forth in Table 5.

TABLE 5

| | |
|---|---|
| Component | Wt. % |

Phase A

| | |
|---|---|
| PEG-20 Methyl Glucose | 0.25 |
| Sesquistearate (Glucamate ™ | |
| SSE-20 Emulsifier, Lubrizol | |
| Advanced Materials, Inc.) | |
| Glyceryl Stearate (emulsifier) | 1.0 |
| Stearamidopropyl | 1.0 |
| Dimethylamine (surfactant) | |
| Isostearyl Hydroxystearate | 2.0 |
| (Schecemol ™ SHS emollient, | |
| Lubrizol Advanced Materials, | |
| Inc.) | |
| Isostearyl Isostearate | 3.0 |
| (Schercemol ™ 1818 emollient, | |
| Lubrizol Advanced Materials, | |
| Inc.) | |
| Diisopropyl Sebacate | 4.0 |
| (Schercemol ™ DIS emollient, | |
| Lubrizol Advanced Materials, | |
| Inc.) | |
| Cetearyl alcohol(emulsifier) | 4.0 |

Phase B

| | |
|---|---|
| Sodium EDTA (chelating agent) | 0.10 |
| Lauryl Methyl Gluceth-10 | 1.00 |
| Hydroxypropyldimonium | |
| chloride (Gluquat ™ 125 | |
| humectant, Lubrizol Advanced | |
| Materials, Inc.) | |
| Sorbitol (70%) (humectant) | 2.50 |
| Methyl gluceth-20 (Glucam ™ | 3.00 |
| E-20 humectant, Lubrizol | |
| Advanced Materials, Inc.) | |
| Aqua (deionized water) | q.s. to 100 |

Phase C

| | |
|---|---|
| Polyquaternium-47 | 1.0 |
| (Merquat ™ 2001 Conditioning | |
| agent, Lubrizol Advanced | |
| Materials, Inc.) | |
| Aqua (deionized water) | 3.0 |

Phase D

| | |
|---|---|
| Methylchloroisothiazoline (and) | 0.05 |
| Methylisothiazoline (Euxyl ™ | |
| K120 preservative, Schülke Inc) | |

Phase E

| | |
|---|---|
| Citric Acid 50% (pH adjusting | 0.45¹ |
| agent 50% aqueous wt./wt.)¹ | |

¹pH of the total composition 3.2-3.8

The components of Phases A and B were combined in separate containers and heated to 70-75° C. while stirring. Phases A and B were combined and mixed until homogeneous while the temperature was maintained at 70-75° C. Phase AB was allowed to cool to 50° C. while stirring. Phase C was prepared by dissolving the Polyquarternium-47 component in deionized water. Phase C was then combined into Phase AB while stirring and maintaining the temperature at 50° C. Phase D was then added to Phase ABC and mixed until homogeneous. Phase E was added to adjust the final pH of the composition.

Following the masque treatment each of the straightened hair tresses were evaluated for shampoo wash-fastness following the protocol described under the solution dipping test. The results are set forth in Table 6.

TABLE 6

| | | | | Flat | | | |
|---|---|---|---|---|---|---|---|
| | EC | PC | | Iron | | | |
| Ex. | (wt. | (wt. | L-Arginine | Speed | Shampoo | Type | |
| No. | %) | %) | (wt. %) | (cm/s) | Cycles | Rating⁴ | Evaluation |
| 18 | 10.0 | 3.3 | 5.0 | 2.3 | 3 | IV | C |
| 19 | 12.0 | 4.0 | 3.0 | 2.3 | 24 | II | R |
| 20 | 12.0 | 4.0 | 8.0³ | 2.3 | 24 | II | R |
| 21 | 14.0 | 4.7 | 6.0 | 2.3 | 30 | II | R |
| 22 | 16.0 | 5.3 | 4.0 | 2.3 | 30 | I | S |
| 23 | 16.0 | 5.3 | 4.0 | 4.6 | 3 | IV | C |
| 24 | 16.5 | 5.5 | 8.5³ | 2.3 | 30 | I | S |
| 25 | 18.0 | 6.0 | 7.0 | 2.3 | 30 | I | S |
| 26 | 20.0 | 6.7 | 5.0 | 2.3 | 24 | I | S |
| 27 | 20.0 | 6.7 | 5.0 | 2.3 | 30 | I | S |
| 28 | 22.0 | 7.3 | 3.0 | 2.3 | 24 | I | S |
| 29 | 25.0 | 8.3 | 5.0 | 2.3 | 30 | I | S |
| 30 | 27.0 | 9.0 | 3.0 | 2.3 | 30 | I | S |
| 31^{1, 2} | 0 | 0 | 0 | 2.3 | 1 | IV | C |
| 32¹ | 15.0 | 5.0 | 0 | 2.3 | 30 | IV | C |
| 33¹ | 20.0 | 6.7 | 0 | 2.3 | 30 | IV | C |
| 34¹ | 25.0 | 8.3 | 0 | 2.3 | 30 | I-II | S |
| 35¹ | 30.0 | 10 | 0 | 2.3 | 30 | I | S |

¹Comparative
²Cream base only (no actives)
³Cream base contained 11.0 wt. % L-arginine
⁴Classified according to Shape Variability and Classification of Human Hair: A Worldwide Approach, Human Biology, vol. 79, No. 3 June 2007.
EC = Ethylene Carbonate
PC = Propylene Carbonate
C = Curley
R = Relaxed
S = Straight

The results demonstrate that the straightening can be achieved by selection of contact time, glide rate, and concentration of thermally activated agents. Including a guanidine moiety containing compound allows the formulator to use significantly less of the hair straightening active ingredient (e.g., ethylene carbonate). Lower concentrations of active ingredients can be utilized to achieve relaxed hair configurations. As is shown from a comparison of experiment nos. 5 and 6, glide rate is an important factor in achieving a straight hair configuration.

An adjuvant cream containing creatinine was formulated from the components and amounts of set forth in Table 7.

TABLE 7
(Part 1 Adjuvant Cream)

| | | |
|---|---|---|
| Component | INCI Name | Wt. % |

Phase A

| | | |
|---|---|---|
| 1 | Ceteareth-20 (emulsifier) | 0.3 |
| 2 | Petrolatum (emollient) | 1.5 |
| 3 | Methyl Glucose Sesquistearate | 1.7 |
| | (Glucate ™ SS Emulsifier, | |
| | Lubrizol Advanced Materials, | |
| | Inc.) | |
| 4 | Glyceryl Stearate (emulsifier) | 2.0 |
| 5 | Parafinium Liquidium | 2.0 |
| | (emollient) | |
| 6 | Cetearyl Alcohol¹ | 4.5 |
| | (emulsifier/conditioning agent) | |

Phase B

| | | |
|---|---|---|
| 7 | Aqua (deionized water diluent) | q.s. to 100 |
| 8 | Methyl Gluceth-20 (Glucam ™ | 1.5 |
| | E-20 Humectant, Lubrizol | |
| | Advanced Materials, Inc.) | |
| 9 | Creatinine | 7.5 |

Phase C

| | | |
|---|---|---|
| 10 | Methylchloroisothiazoline (and) | 0.05 |
| | Methylisothiazoline (Euxyl ™ | |
| | K120 Preservative, Schülke | |
| | Inc.) | |
| 11 | Cyclopenasiloxane (Dow | 2.0 |
| | Corning ™ DC 245 | |
| | decamethylcyclopentasiloxane | |
| | conditioning agent) | |

Phase D

| | | |
|---|---|---|
| 12 | Polyquaternium-47 | 0.5 |
| | (Merquat ™ 2001 Conditioning | |
| | agent, Lubrizol Advanced | |
| | Materials, Inc.) | |
| 13 | Aqua (deionized water diluent) | 2.0 |

¹30/70 (wt./wt.) blend of Cetyl and Stearyl Alcohols
pH of the composition = 6.5-7.0

The components of Phases A and B were combined in separate containers and heated to 70-75° C. while stirring. Phases A and B were combined and mixed until homogeneous while the temperature was maintained at 70-75° C. Phase AB was allowed to cool to 50° C. while stirring. Phase C was prepared by dissolving the Polyquarternium-47 component in deionized water. Phase C was then combined into Phase AB while stirring and maintaining the temperature at 50° C. Phase D was then added to Phase ABC and mixed until a homogeneous creamy emulsion was obtained.

A cream hair straightening formulation was prepared by homogeneously mixing the Phase A with the Phase B components set forth in Table 8.

TABLE 8

| | |
|---|---|
| Component | Wt. % |

Phase A

| | | |
|---|---|---|
| Adjuvant Cream | 40 | 66.67 |
| (7.5 wt. % | | |
| Creatinine) | | |

Phase B

| | | |
|---|---|---|
| EC/PC Solution | 36.0 | 33.33 |
| (75 wt. % EC | | |
| Aqua (deionized | 24.0 | — |
| water diluent) | | |

EC = Ethylene Carbonate
PC = Propylene Carbonate

Hair tresses were prepared, treated and evaluated as set forth in Examples 18-35 above. The results are presented in Table 9.

TABLE 9

| | | | | Flat | | | |
|---|---|---|---|---|---|---|---|
| | EC | PC | | Iron | | | |
| Ex. | (wt. | (wt. | | Speed | Shampoo | Type | |
| No. | %) | %) | Creatinine | (cm/s) | Cycles | Rating³ | Evaluation |
| 36 | 27.0 | 9 | 3.0 | 2.3 | 24 | I | S |
| 37 | 25.0 | 8.3 | 5.0 | 2.3 | 24 | I | S |

An adjuvant cream containing creatine ethyl ester hydrochloride was formulated from the components and amounts of set forth in Table 10.

TABLE 10
(Part 1 Adjuvant Cream)

| | | |
|---|---|---|
| Component | INCI Name | Wt. % |

Phase A

| | | |
|---|---|---|
| 1 | Ceteareth-20 (emulsifier) | 0.3 |
| 2 | Petrolatum (emollient/emulsifier) | 1.5 |
| 3 | Methyl Glucose Sesquistearate | 1.7 |
| | (Glucate ™ SS Emulsifier, Lubrizol | |
| | Advanced Materials, Inc.) | |
| 4 | Glyceryl Stearate (emulsifier) | 2.0 |
| 5 | Parafinium Liquidium (emollient) | 2.0 |
| 6 | Cetearyl Alcohol¹ | 4.5 |
| | (emulsifier/conditioning agent) | |

Phase B

| | | |
|---|---|---|
| 7 | Aqua (deionized water diluent) | q.s. to 100 |
| 8 | Methyl Gluceth-20 (Glucam ™ | 1.5 |
| | E-20 Humectant, Lubrizol Advanced | |
| | Materials, Inc.) | |
| 9 | Sodium Hydroxide (18% aqueous | 9.2 |
| | wt./wt.) | |

Phase C

| | | |
|---|---|---|
| 10 | Creatine Ethyl Ester HCl | 7.5 |
| 11 | Aqua (deionized water diluent) | 30.0 |

Phase D

| | | |
|---|---|---|
| 12 | Methylchloroisothiazoline (and) | 0.05 |
| | Methylisothiazoline (Euxyl ™ K120 | |
| | Preservative, Schülke Inc.) | |
| 13 | Cyclopenasiloxane (Dow | 2.0 |
| | Corning ™ DC 245 | |
| | decamethylcyclopentasiloxane | |
| | conditioning agent) | |

Phase E

| | | |
|---|---|---|
| 14 | Polyquaternium-47 | 0.5 |
| | (Merquat ™ 2001 Conditioning | |
| | agent, Lubrizol Advanced | |
| | Materials, Inc.) | |
| 15 | Aqua (deionized water diluent) | 2.0 |

¹30/70 (wt./wt.) blend of Cetyl and Stearyl Alcohols
pH of the composition = 6.5-7.0

The components of Phases A and B were combined in separate containers and heated to 70-75° C. while stirring. Phases A and B were combined and mixed until homogeneous while the temperature was maintained at 70-75° C. Phase AB was allowed to cool to 50° C. while stirring. The Phase C components were mixed until homogeneous and then mixed into the Phase AB emulsion while stirring and maintaining the temperature at 50° C. Phase D was then added to Phase ABC emulsion and mixed until homogeneous. Phase E was prepared by dissolving the Polyquaternium-47 component in deionized water and then was added to the ABCD emulsion and mixed until a homogeneous creamy emulsion was obtained.

A cream hair straightening formulation was prepared by homogeneously mixing the Phase A with the Phase B components set forth in Table 11.

TABLE 11

| | |
|---|---|
| Component | Wt. % |

Phase A

| | | |
|---|---|---|
| Adjuvant Cream | 40 | 66.67 |
| (7.5 wt. % Creatine | | |
| Ethyl Ester HCl) | | |

Phase B

| | | |
|---|---|---|
| EC/PC Solution | 36.0 | 33.33 |
| (75 wt. % EC | | |
| Aqua (deionized | 24.0 | — |
| water diluent) | | |

EC = Ethylene Carbonate
PC = Propylene Carbonate

Hair tresses were prepared, treated and evaluated as set forth in Examples 18-37 above. The results are presented in Table 12.

TABLE 12

| | | | | Flat | | | |
|---|---|---|---|---|---|---|---|
| | EC | PC | | Iron | | | |
| Ex. | (wt. | (wt. | | Speed | Shampoo | Type | |
| No. | %) | %) | Creatinine | (cm/s) | Cycles | Rating³ | Evaluation |
| 38 | 27.0 | 9 | 3.0 | 2.3 | 24 | I | S |
| 39 | 25.0 | 8.3 | 5.0 | 2.3 | 24 | I | S |

Except in the Examples, or where otherwise explicitly indicated, all numerical quantities in this description specifying amounts of materials, reaction conditions, molecular weights, number of carbon atoms, and the like, are to be understood as modified by the word “about.” Unless otherwise indicated, each chemical or composition referred to herein should be interpreted as being a commercial grade material which may contain the isomers, by-products, derivatives, and other such materials which are normally understood to be present in the commercial grade. However, the amount of each chemical component is presented exclusive of any solvent or diluent oil, which may be customarily present in the commercial material, unless otherwise indicated. It is to be understood that the upper and lower amount, range, and ratio limits set forth herein may be independently combined. Similarly, the ranges and amounts for each element of the disclosed technology may be used together with ranges or amounts for any of the other elements. As used herein, the expression “consisting essentially of” permits the inclusion of substances that do not materially affect the basic and novel characteristics of the composition under consideration. As used herein any member of a genus (or list) may be excluded from the claims.

It will be appreciated that variants of the above-disclosed and other features and functions, or alternatives thereof, may be combined into many other different systems or applications. Various presently unforeseen or unanticipated alternatives, modifications, variations or improvements therein may be subsequently made by those skilled in the art which are also intended to be encompassed by the following claims.

1. A composition for straightening or relaxing hair comprising a thermally-activated composition, said composition comprising:
(a) ethylene carbonate as the hair straightening agent; and
(b) at least one hair straightening adjuvant compound selected from a guanidine derivative and/or the organic and inorganic salts thereof selected from a guanidine moiety containing compound represented by the radical: 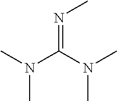
wherein the composition is free of all compounds which contain thiol groups.

2. (canceled)

3. A composition of claim 1, wherein said guanidine moiety containing compound is represented by Formula (II): 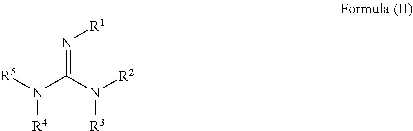
wherein R¹, R², R³, R⁴ and R⁵ independently represent a hydrogen atom; linear or branched C₁ to C₈ alkyl; linear or branched C₂ to C₄ alkenyl; phenyl; benzyl; wherein said linear or branched C₁-C₄ alkyl said linear or branched C₂ to C₄ alkenyl, said phenyl and said benzyl groups can optionally be substituted with one or two substituents selected from halo, amino, dimethylamino, carboxyl, hydroxyl, methyl, methoxy, carboxamide, N-methylcarboxamide and —SO₃H; and any two of R¹, R², R³, R⁴ and R⁵ together with the nitrogen atom(s) to which they are attached form a 4 to 9 membered monocyclic or bicyclic ring containing 2 to 4 carbon atoms, optionally containing one or two (ring) heteroatom(s) selected from C(O), N, O and S, and said carbon atom(s) being optionally substituted with one or two substituent(s) selected from hydroxyl and amino;
when R¹ represents a hydrogen atom, —C(O)OC(CH₃)₃, or the radical 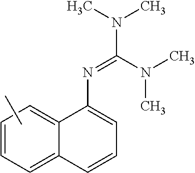
R², R³, R⁴, and R⁵ can independently represent a hydrogen atom, a radical selected from amino, nitro; cyano; acetyl; chloroacetyl; carboxyl, carboxamide; N-methylcarboxamide; methoxy; ethoxy; 1,2,4-triazolyl; cyclopentyl; cyclohexyl; —C(O)CH═CHC(O)OH; —CH₂(CO)OCH₃; —CH₂C(O)OCH₂CH₃; —C(O)OC(CH₃)₃; thiazolidonyl; benzimidazolyl; benzoxazolyl; benzothiazolyl; —C(═NH)NH₂; —C(═NH)—NR⁶R⁷ where R⁶ and R⁷ independently represent a hydrogen atom or a linear or branched C₁-C₄ alkyl radical, optionally substituted with one or 2 substituents selected from hydroxyl, amino, dimethylamino, carboxyl and carboxamide; or 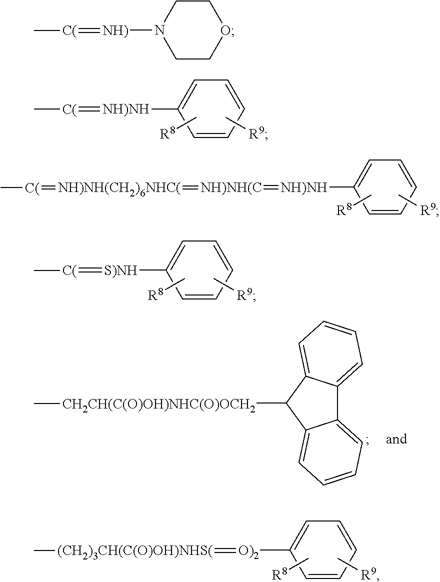
where R⁸ and R⁹ independently represent halo, methyl, methoxy, trifluoromethyl, and trifluoromethoxy; and
optionally
(c) at least one cosmetically acceptable excipient; and
wherein said hair straightening agent is present in an amount ranging from about 10 to about 70 wt. % (based on the total weight of the composition), and the ratio of said hair straightening agent to said adjuvant ranges from about 1.5 to 1 to about 10:1 by weight.

4. A composition of claim 1, wherein said hair straightening agent is selected from a mixture of ethylene carbonate and propylene carbonate.

5. (canceled)

6. A composition of claim 3, wherein in said Formula (II) R¹, R² and R³ represent a hydrogen atom; and R⁴ and R⁵ taken together with the nitrogen atom to which they are attached, represents a pyrrolidine, piperidine, pyrazole, thiazolidone, benzimidazole, benzoxazole, benzothiazole, or 1,2,4-triazole ring, said rings being optionally substituted with 1 or 2 substituents selected from hydroxyl, amino and carboxyl groups.

7. A composition of claim 3, wherein in Formula (II) R¹ and R² represent a hydrogen atom, and R⁵ represents a hydrogen atom or methyl, R³ and R⁴ taken together can represent a divalent radical selected from —CH₂—CH₂— and —CH₂—O— and when taken together with the nitrogen atoms to which they are attached represent a 5 membered ring.

8. A composition of claim 3, wherein said adjuvant is selected from at least one compound represented by the Formula (IIA): 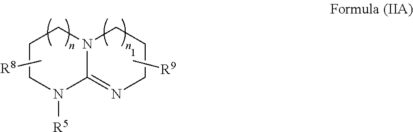
where R⁵ is as defined above; R⁸ and R⁹ are optionally present, and when present one or two of R⁸ and R⁹ can be bonded to the same or different ring carbon atom and independently represent C₁-C₅ alkyl, hydroxyl, amino, dimethylamino, carboxyl, carboxamide, N-methylcarboxamide and SO₃H; n and n₁ independently represent 0, 1 or 2.

9. A composition of claim 3, wherein said adjuvant is selected from at least one compound represented by Formulas (IIB), (IIC), (IID), (IIE) and (IIF) as follows: 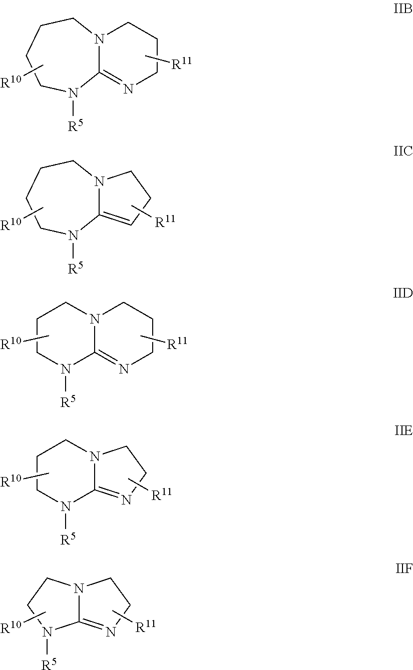
where R⁵, R⁸ and R⁹ are as previously defined.

10. A composition of claim 9, wherein R⁵ represents a hydrogen atom and R⁸ and R⁹ are not present.

11. A composition of claim 1, wherein said adjuvant is selected from guanidine; guanidine hydrochloride; guanidine acetate; guanidine sulfate; guanidine carbonate; guanidine bicarbonate; guanidine nitrate; guanidine phosphate; guanidine sulfamate; aminoguanidine; aminoguanidine hydrochloride; aminoguanidine nitrate; aminoguanidine sulfate; aminoguanidine bicarbonate; 1,3-diaminoguanidine hydrochloride; cyanoguanidine; nitroguanidine; 1-methyl-3-nitroguanidine; 1-acetylguanidine; chloroacetylguanidine hydrochloride; guanidine acetic acid guanylurea; guanylurea phosphate; phenylguanidine carbonate; phenylguanidine bicarbonate; 1,3-diphenylguanidine; 1,3-di-o-tolylguanidine; methyl 4-guanidino-2-methoxybenzoate hydrochloride; 1-methylguanidine hydrochloride; 1,1-dimethylguanidine hydrochloride; 1,1-dimethylguanidine sulfate; 1-ethylguanidine hydrochloride; 1-ethylguanidine sulfate; 1-octylguanidine hemisulfate; 1,1-diethylguanidine hydrochloride; N-tert-butoxycarbonylguanidine (N-Boc guanidine) 1,3-bis(tert-butoxycarbonyl)guanidine (Di-Boc guanidine); carbamic acid; N,N-[[(1,1-dimethylethoxy)carbonyl]carbonimidoyl]bis-; C,C′-bis(1,1-dimethylethyl) ester (N,N′,N″-Tri-Boc guanidine); creatine; creatine monohydrate; creatinine; creatinine hydrochloride; creatine ethyl ester; agmatine; agmatine sulfate; guanidineacetic acid; guanidinosuccinic acid; 3-guanidinopropionic acid; 4-guanidinobutyric acid; 5-guanidinovaleric acid; 4-guanidine benzoic acid; 4-guanidine benzoic acid hydrochloride; 4-guanidino-2-methoxybenzoic acid; beta-N-methylguanidinopropionic acid; N-methylguanidinopropionic acid; N-(2-hydroxyethyl)guanidine; N-(3-hydroxypropyl)guanidine; biguanide hydrochloride; N-methylbiguanide hydrochloride; N-ethylbiguanide hydrochloride; N-propylbiguanide hydrochloride; N-butylbiguanide hydrochloride; 1-o-tolylbiguanide; 1,1-dimethylbiguanide hydrochloride; 1-(3-chloro-4-fluorophenyl)biguanide hydrochloride; 1-(2-chlorophenyl)biguanide hydrochloride; 1-(4-chlorophenyl)biguanide hydrochloride; 1-(2-fluorophenyl)biguanide hydrochloride; 1-(3-fluorophenyl)biguanide hydrochloride; 1-(4-fluorophenyl)biguanide hydrochloride; 1-(2,3-dichlorophenyl)biguanide hydrochloride; 1-(2,4-dichlorophenyl)biguanide hydrochloride; 1-(2,5-dichlorophenyl)biguanide hydrochloride; 1-(2,6-dichlorophenyl)biguanide hydrochloride; 1-(3,4-dichlorophenyl)biguanide hydrochloride; 1-(3,5-dichlorophenyl)biguanide hydrochloride; 1-(2,4-difluorophenyl)biguanide hydrochloride; 1-(2,5-difluorophenyl)biguanide hydrochloride; 1-[4-(trifluoromethyl)phenyl]biguanide hydrochloride; 1-[3,5-di-(trifluoromethyl)phenyl]biguanide hydrochloride; 1-[4-(trifluoromethoxy)phenyl]biguanide hydrochloride; 1-phenylbiguanide; 1-phenylbiguanide carbonate; 1,1′-hexamethylenebis[5-(4-chlorophenyl)biguanide]; 1,1′-hexamethylenebis(5-[p-chlorophenyl]biguanide) dihydrochloride; [({[Amino(imino)methyl]amino}carbothioyl)amino]benzene; N-cyclohexyl guanidine hydrochloride; N-benzylguanidine hydrochloride; N-benzyl-N-methylguanidine hemisulfate; 1,1,3,3-tetramethylguanidine; 1,1,3,3-tetramethylguanidine hydrochloride; 2-tert-butyl-1,1,3,3-tetramethylguanidine; 2-tert-butyl-1,1,3,3-tetramethylguanidine hydrochloride; 4-morpholinecarboximidoylguanidine hydrochloride; L-arginine; D-arginine; DL-arginine; arginic acid; N-tosyl-L-Arginine; N-amidino-N-(2,3-dihydroxypropyl)glycine; N-amidinotaurine; 2-imino-1-imidazolidineacetic acid; 1-(2,2-diethoxyethyl)guanidine; (2S)-3-carbamimidamido-2-{[(9H-fluoren-9-ylmethoxy)carbonyl]amino}propanoic acid; 1,8-bis(tetramethylguanidino)naphthalene; 1H-pyrazole-1-carboxamidine hydrochloride; 5-hydroxy-3-methyl-1H-pyrazole-1-carboximidamide; 3,5-diamino-1H-1,2,4-triazole-1-carboximidamide hydrochloride; 2-guanidone-4-thiazolidone; 2-guanidinobenzimidazole; 2-guanidinobenzoxazole; 2-guanidinobenzothiazole; pyrrolidinoformamidine hydrochloride; 2,3,4,6,7,8,9,10-octahydropyrimido[1,2-a][1,3]diazepine (Htbu); 2,5,6,7,8,9-hexahydro-3H-imidazo[1,2-a][1,3]diazepine (Htbd); 1,5,7-triazabicyclo[4.4.0]dec-5-ene (Hhpp); 3,3,9,9-tetramethyl-1,2,7,triazabicyclo[4.4.0]dec-5-ene (HTMhpp); 3,3,9,9-tetraethyl-1,2,7-triazabicyclo[4.4.0]dec-5-ene (HTEhpp); 1,5,7-triazabicyclo[4.3.0]non-6-ene (Htbn); 1,4,6-triazabicyclo[3.3.0]oct-4-ene (Htbo); and mixtures thereof.

12. A composition of claim 4, wherein said hair straightening agent (a) is selected from a mixture of ethylene carbonate and propylene carbonate present in a wt. ratio of ethylene carbonate to propylene carbonate ranging from about 95:5 to about 5:95; and said adjuvant (b) is a guanidine moiety containing compound selected from guanidine, guanidine hydrochloride, guanidine bicarbonate, 3-guanidinopropionic acid, creatinine, creatine, creatine ethyl ester, L-arginine, 1,5,7-triazabicyclo[4.4.0]dec-5-ene, and mixtures thereof and wherein the ratio of said hair straightening agent to said adjuvant ranges from about 1.5 to 1 to about 10:1 by weight.

13. A composition of claim 3, wherein said at least one cosmetically acceptable excipient (c) is selected from preservatives, antioxidants, chelating agents, sunscreen agents, vitamins, proteins, amino acids, plant extracts, humectants, fragrances, perfumes, emollients, surfactants, penetrants, thickening agents, viscosity modifiers, emulsifiers, opacifying agents, propellants, diluents, pH adjusting agents, buffers, conditioning agents, anti-static agents, anti-frizz agents, and mixtures thereof.

14. A composition of claim 13, wherein the diluent is selected from water, propylene carbonate, alcohol, and mixtures thereof.

15. A composition of claim 3, wherein the composition comprises a compound selected from surfactant(s), emulsifier(s), emollient(s), humectant(s) conditioning agent(s), thickening agent(s), and mixtures thereof.

16. (canceled)

17. (canceled)

18. A system for straightening hair comprising at least two separate components:
i) a first component (a) comprising ethylene carbonate as the hair straightening agent, wherein said first component does not contain a guanidine moiety containing compound (b);
ii) a second component comprising at least one hair straightening adjuvant selected from a guanidine moiety containing compound (b) as described in claim 11, wherein said second component does not contain said hair straightening compound (a); and
iii) wherein said first component (a) and said second component (b) independent of the other optionally contain a cosmetically acceptable excipient (c) as described in claim 13;
wherein said first hair component (a) and said second component (b) are suitable for being mixed together, and after mixing said hair straightening agent is present in an amount ranging from about 10 to about 70 wt. % (based on the total weight of the composition), and the ratio of said hair straightening agent to said adjuvant ranges from about 1.5 to 1 to about 10:1 by weight,
wherein said components are free of all compounds which contain thiol groups.

19. A system of claim 18, wherein said hair straightening agent is selected from a mixture of ethylene carbonate and propylene carbonate.

20. (canceled)

21. A system of claim 18, wherein said guanidine moiety containing compound (b) is selected from guanidine, guanidine hydrochloride, guanidine acetate, guanidine sulfate, guanidine carbonate, guanidine bicarbonate, guanidine phosphate, guanidine sulfamate, aminoguanidine, inoguanidine hydrochloride, aminoguanidine sulfate, aminoguanidine bicarbonate, 1,3-diaminoguanidine hydrochloride, 1-acetylguanidine, chloroacetylguanidine hydrochloride, guanylurea, guanylurea phosphate, phenylguanidine carbonate, phenylguanidine bicarbonate, 1-methylguanidine hydrochloride, 1,1-dimethylguanidine hydrochloride, 1-ethylguanidine hydrochloride, 1,1-diethylguanidine hydrochloride, creatine, creatine monohydrate, creatinine, creatinine hydrochloride, creatine ethyl ester, agmatine, agmatine sulfate, guanidinoacetic acid, guanidinosuccinic acid, 3-guanidinopropionic acid, 4-guanidinobutyric acid, 5-guanidinovaleric acid, beta-N-methylguanidinopropionic acid, N-methylguanidinopropionic acid, N-(2-hydroxyethyl)guanidine, N-(3-hydroxypropyl)guanidine, biguanide hydrochloride, N-methylbiguanide hydrochloride, N-ethylbiguanide hydrochloride, N-propylbiguanide hydrochloride, N-butylbiguanide hydrochloride, 1,1-dimethylbiguanide hydrochloride, 1-phenylbiguanide, 1,1,3,3-tetramethylguanidine hydrochloride, 2-tert-butyl-1,1,3,3-tetramethylguanidine hydrochloride, L-arginine, D-arginine, DL-arginine, arginic acid, N-amidino-N-(2,3-dihydroxypropyl)glycine, N-amidinotaurine, 2-imino-1-imidazolidineacetic acid, 1-(2,2-diethoxyethyl)guanidine, 1H-pyrazole-1-carboxamidine hydrochloride, 5-hydroxy-3-methyl-1H-pyrazole-1-carboximidamide, 3,5-diamino-1H-1,2,4-triazole-1-carboximidamide hydrochloride, 2-guanidone-4-thiazolidone, 2-guanidinobenzimidazole, 2-guanidinobenzoxazole, 2-guanidinobenzothiazole, pyrrolidinoformamidine hydrochloride, 2,3,4,6,7,8,9,10-octahydropyrimido[1,2-a][1,3]diazepine (Htbu), 2,5,6,7,8,9-hexahydro-3H-imidazo[1,2-a][1,3]diazepine (Htbd), 1,5,7-triazabicyclo[4.4.0]dec-5-ene (Hhpp), 3,3,9,9-tetramethyl-1,2,7,triazabicyclo[4.4.0]dec-5-ene (HTMhpp), 3,3,9,9-tetraethyl-1,2,7-triazabicyclo[4.4.0]dec-5-ene (HTEhpp), 1,5,7-triazabicyclo[4.3.0]non-6-ene (Htbn), 1,4,6-triazabicyclo[3.3.0]oct-4-ene (Htbo), and/or mixtures thereof.

22. A system of 18, wherein said guanidine moiety containing compound (b) is selected from guanidine, guanidine hydrochloride, guanidine bicarbonate, 3-guanidinopropionic acid, creatinine, creatine, creatine ethyl ester, L-arginine, 1,5,7-triazabicyclo[4.4.0]dec-5-ene, and/or mixtures thereof.

23. (canceled)

24. (canceled)

25. A system of claim 18, wherein said first component (a) and said second component (b) upon mixing together form a thermally-activatable hair straightening composition.

26. A system of claim 25, wherein said thermally-activatable hair straightening composition is in the form of a cream.

27. A kit for preparing a thermally-activatable composition for straightening hair, the kit comprising:
i) a hair straightening component (a) contained in a first container, said component comprising ethylene carbonates as the hair straightening agent, wherein said first component (a) does not include a guanidine moiety containing compound;
ii) a hair straightening adjuvant component (b) contained in a second container, said component comprising at least one adjuvant selected from a guanidine moiety containing compounds described in claim 11, wherein said second component (b) does not include said hair straightening agent; and
iii) wherein said first component (a) and said second component (b) independent of the other optionally contain a cosmetically acceptable excipient (c) as described in claim 13;
wherein said first hair straightening agent component (a) and said second adjuvant component (b) are suitable for being mixed and after mixing said hair straightening agent is present in an amount ranging from about 10 to about 70 wt. % (based on the total weight of the composition), and the ratio of said hair straightening agent to said hair straightening adjuvant ranges from about 1.5 to 1 to about 10:1 by weight;
wherein said composition is free of all compounds which contain thiol groups, and
wherein said containers are packaged together in kit form for use.

28. A kit of claim 27, wherein said hair straightening agent is selected from a mixture of ethylene carbonate and propylene carbonate.

29. (canceled)

30. (canceled)

31. A kit of claim 27, wherein said guanidine moiety containing compound (b) is selected from guanidine, guanidine hydrochloride, guanidine bicarbonate, 3-guanidinopropionic acid, creatinine, creatine, creatine ethyl ester, L-arginine, 1,5,7-triazabicyclo[4.4.0]dec-5-ene, and/or mixtures thereof.

32. (canceled)

33. (canceled)

34. A kit of claim 27, wherein said first composition and said second composition upon mixing form a thermally-activatable hair straightening composition.

35. A kit of claim 34, wherein said thermally-activatable hair straightening composition is in the form of a cream.

36. A method for straightening hair comprising:
(i) providing a thermally-activatable hair straightening composition as set forth in claim 1;
(ii) coating hair fibers with said thermally-activatable composition; and
(iii) contacting the coated hair fibers with a heating device set to a temperature of at least 185° C. for sufficient time to modify the keratin fibers.

37. A method for straightening hair comprising:
(i) providing a thermally-activatable hair straightening composition as set forth in any of the previous claims;
(ii) coating hair fibers with said thermally-activatable composition;
(iii) allowing the coated fibers to rest for at least 15 to 60 minutes;
(iv) combing the coated hair fibers from root to end;
(v) drying the combed hair fibers;
(vi) contacting the coated hair fibers with a heating device set to a temperature of at least 185° C. for sufficient time to modify the keratin fibers;
(vii) applying a masque composition to the heat treated hair fibers;
(viii) rinsing the conditioned hair fibers;
(ix) optionally shampooing and rinsing said conditioned hair fibers; and
(xi) drying the straightened hair.

38. A method of claim 36 a temperature ranging from about 185 to 250° C.

39. A method of claim 36, wherein said heating device is pulled through the same hair fibers from the root to the tip from at least 5 to at least 10 repetitions.

40. A method of claim 39, wherein said heating device is pulled through the hair fibers at glide rate of about 1 cm/sec. to about 4 cm/sec.

41. A method of claim 37, wherein the pH of the hair masque ranges from about 2 to about 5.

42. (canceled)

43. (canceled)

44. (canceled)

45. A thermally-activated composition for straightening or relaxing hair comprising:
(a) ethylene carbonate; and
(b) an adjuvant compound selected from guanidine, L-arginine, guanidinopropionic acid, creatinine, creatine, creatine ethyl ester, 1,5,7-triazabicyclo[4.4.0]dec-5-ene; and the salts thereof and mixtures thereof, wherein the amount of ethylene carbonate in said composition ranges from about 10 to about 35 wt. % (based on the total weight of the composition), and the weight ratio of ethylene carbonate to the adjuvant compound in said composition ranges from about 2:1 to about 10:1.

46. A thermally-activated composition of claim 45, further comprising propylene carbonate, wherein the weight ratio of ethylene carbonate to propylene carbonate is from about 80:20 to about 20:80.

47. A thermally-activated composition of claim 45, wherein the salt of the adjuvant compound is selected from the hydrochloride, nitrate, phosphate, sulfate, hemisulfate, sulfamate, acetate, carbonate, bicarbonate, and monohydrate salt, and mixtures thereof.

48. A thermally-activated composition of claim 45, wherein said adjuvant compound is selected from L-arginine, guanidine hydrochloride, guanidine carbonate, guanidinopropioic acid, creatinine, creatine ethyl ester hydrochloride, 1,5,7-triazabicyclo[4.4.0]dec-5-ene, and mixtures thereof.

49. A thermally-activated composition of claim 45, further comprising (c) a cosmetically acceptable excipient.

50. A thermally-activated composition of claim 49, wherein said cosmetically acceptable excipient is selected from preservatives, antioxidants, chelating agents, proteins, humectants, fragrances, perfumes, emollients, surfactants, thickening agents, viscosity modifiers, emulsifiers, opacifying agents, propellants, diluents, pH adjusting agents, buffers, conditioning agents, and mixtures thereof.

51. A thermally-activated composition of claim 50, wherein said conditioning agent is selected from a silicone, a cationic compound, a cationic polymer, a hydrocarbon oil, a natural oil, a natural wax, a synthetic wax, and mixtures thereof.

52. A thermally-activated composition of claim 50, wherein said conditioning agent is a cationic polymer selected from Polyquaternium-4, Polyquaternium-6, Polyquaternium-10, Polyquaternium-11, Polyquarternium-16, Polyquaternium-24, Polyquaternium-28, Polyquaternium-29, Polyquaternium-32, Polyquaternium-33, Polyquaternium-35, Polyquaternium-37, Polyquaternium-44, Polyquaternium-46, Polyquaternium-47, Polyquaternium-52, Polyquaternium-53, Polyquarternium-55, Polyquaternium-59, Polyquaternium-61, Polyquaternium-64, Polyquaternium-65, Polyquaternium-67, Polyquaternium-69, Polyquaternium-70, Polyquaternium-71, Polyquaternium-72, Polyquaternium-73, Polyquaternium-74, Polyquaternium-76, Polyquaternium-77, Polyquaternium-78, Polyquaternium-79, Polyquaternium-80, Polyquaternium-81, Polyquaternium-82, Polyquaternium-84, Polyquaternium-85, Polyquaternium-87, and PEG-2-cocomonium chloride, Starch Hydroxypropyltrimonium Chloride, Guar Hydroxypropyltrimonium Chloride, Hydroxypropyl Guar Hydroxypropyltrimonium Chloride, and Cassia Hydroxypropyltrimonium Chloride, and mixtures thereof.

53. (canceled)

54. (canceled)

55. (canceled)

56. (canceled)

57. A composition of claim 14, wherein said alcohol is selected from a C₁-C₁₀ aliphatic alcohol.

58. A composition of claim 57, wherein said C₁-C₁₀ aliphatic alcohol is selected from ethylene glycol, propylene glycol, glycerin, and mixtures thereof.

59. A thermally-activated composition of claim 45, further comprising a C₁-C₁₀ aliphatic alcohol.

60. A thermally-activated composition of claim 59, wherein said C₁-C₁₀ aliphatic alcohol is selected from ethylene glycol, propylene glycol, glycerin, and mixtures thereof.
